(19) United States
(12) Patent Application Publication
(10) Pub. No.: US 2015/0038333 A1
(43) Pub. Date: Feb. 5, 2015

(54) PLANT PROTECTIVE COMPOSITION CONTAINING ALPHA-HYDROXY ACIDS

(71) Applicants: Veronica Von Saint Paul, Munchen (DE); Wei Zhang, Munchen (DE); Anton Schäffner, Kirchseeon (DE)

(72) Inventors: Veronica Von Saint Paul, Munchen (DE); Wei Zhang, Munchen (DE); Anton Schäffner, Kirchseeon (DE)

(73) Assignee: HELMHOLTZ ZENTRUM MÜNCHEN DEUTSCHES FORSCHUNGSZENT -RUN FÜR GESUNDHEIT UND, Neuherberg (DE)

(21) Appl. No.: 14/234,902

(22) PCT Filed: Jul. 26, 2012
(86) PCT No.: PCT/EP2012/064723

§ 371 Date: May 12, 2014

(30) Foreign Application Priority Data
Jul. 26, 2011 (EP) 11006125.6

(51) Int. Cl. A01N 37/36; C07C 59/01

(52) U.S. Cl. CPC A01N 37/36; C07C 59/01
USPC 504/320; 562/579

(57) ABSTRACT

The present invention relates to a plant protective composition comprising α-hydroxy-acid or a derivative thereof. The present invention further relates to a method of protecting plants from biotrophic pathogens comprising contacting the plants with the plant protective composition.

The present invention relates to a plant protective composition comprising α-hydroxy-acid or a derivative thereof. The present invention further relates to a method of protecting plants from pathogens comprising contacting the plants with the plant protective composition.

In this specification, a number of documents including patent applications and manufacturer's manuals is cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Plants are sessile organisms and cannot escape adverse environmental cues. They therefore have evolved elaborate mechanisms to antagonize stress and to organize defense or tolerance. These measures involve a complex reprogramming of plant cells, which relies on major changes in gene expression, protein modification and a range of different compounds active in defense and signaling. Several small-molecule hormones such as salicylic acid (SA), jasmonic acid (JA), ethylene, and abscisic acid play crucial roles in regulating responses of plants to both biotic and abiotic stresses. These signaling pathways interact with each other in synergistic as well as antagonistic manners enabling the plant to fine-tune its response to the stressor(s) encountered (Jones and Dangl, 2006; Koornneef and Pieterse, 2008). Constitutive production of signaling molecules and the concomitant expression of defense genes is energetically cost-intensive and re-allocation of resources towards defense is believed to decrease the overall fitness of a plant. Plants therefore need a tight control of the defense response and its suppression in the absence of pathogen attack or other stresses (Heil and Baldwin, 2002; Bolton, 2009).

The main signaling pathways that are triggered when plants defend themselves against pathogens are the SA- and JA-mediated signaling pathways. Although concerted actions of both pathways have been reported, they are usually acting in an antagonistic manner via mutual repression (Jones and Dangl, 2006; Koornneef and Pieterse, 2008; Vlot et al., 2009).

Furthermore, whereas biotrophic pathogens (bacteria, fungi, viruses) are mostly combated by the SA pathway and might be hampered by the activation of the JA response, the opposite prioritization of defense signaling is mobilized to defend against necrotrophic pathogens (bacteria, fungi) and herbivores.

Arabidopsis genetics has defined a plethora of genes involved in both SA and JA signaling, as well as their interplay. A number of mutants were shown to result in an enhanced susceptibility to biotrophic pathogens and a suppression of SA responses, thereby allowing to define crucial steps in SA signaling. These include components of the MAP kinase signaling pathway like ERD1, MPK3 and MPK6, genes related to SA biosynthesis (ICS1/SID2, PAD4, EDS1), central downstream regulators of SA signaling like NPR1, as well as WRKY and TGA transcription factors. Induction of these transcription factors eventually leads to the activation of SA-responsive genes, including PR genes, that are involved in defense responses. Similarly, mutations in e.g. JAR1, COI1, and JIN1, defining different steps in JA signaling, negatively affect the JA pathway (Kazan and Manners, 2008). Resistance towards necrotrophic pathogens is reduced in the corresponding mutants concomitant with the abolished induction of marker genes like the defensin PDF1; 2. In contrast, several gain-of-resistance Arabidopsis mutants show constitutive defense responses in the absence of (biotrophic) pathogen attack affecting pathogen perception and response or leading to primed defense. These mutants are usually characterized by transcriptional activation of PR genes and constitutive accumulation of SA (Vlot et al., 2009).

Plant secondary metabolite UDP-dependent glycosyltransferases (UGTs) catalyze the transfer of a carbohydrate from an activated donor sugar onto small molecule acceptors by the formation of a glycosidic bond (Mackenzie et al., 1997; Bowles et al., 2006). Glycosylation changes the stability and/or solubility of the aglyca and it may even create a higher diversity due to differential and multiple conjugations. These reactions are an important feature of the biosynthesis of many secondary metabolites and in many cases of the regulation of the activity of signaling molecules and defense compounds. They may include detoxification and compartmentalization of endogenous compounds and xenobiotics (Jones and Vogt, 2001). In Arabidopsis thaliana, 122 different UGT isoforms exist, which represent 0.5% of all annotated genes (Bowles et al., 2006). Analyses of recombinant UGT proteins led to the identification of UGTs with in vitro activity towards several endogenous compounds like auxin, ABA, flavonoids, lignin precursors, hydroxybenzoic acids, thiohydroximate, as well as towards xenobiotics (reviewed in Bowles et al., 2006).

However, these activities could be confirmed in vivo only in a few cases possibly due to the broad substrate acceptance of some UGT enzymes in vitro or due to a limited substrate availability in vivo (Jones and Vogt, 2001; Bowles et al., 2006). So far, there is in vivo evidence for Arabidopsis glycosyltransferases conjugating flavonoids, SA, IAA, glucosinolates and brassinosteroids as endogenous substrates (Bowles et al., 2006; Dean and Delaney, 2008; Song et al., 2008). The vast majority of UGT isoforms, however, still remain ‘orphan’ glycosyltransferases without knowledge about their substrates.

US 2009/0018019 describes a method of increasing the inherent defense mechanisms of plants by providing the compound chloronicotinyl, which results in the increased expression of genes encoding PR proteins. A different approach to activating a plant's own defense mechanisms against pathogens is described in U.S. Pat. No. 4,931,581, where derivatives or 7-cyano-1,2,3-benzothiadiazole or 1,2,3-benzothiadiazole-7-carboxylic acid are employed for immunizing plants against attack by diseases.

Despite the fact that a lot of effort has been invested into the elucidation of plant protective mechanisms, there is still a need to provide means and methods to enhance the protective activity of plants against pathogen attacks.

This need is addressed by the provision of the embodiments characterized in the claims.

Accordingly, the present invention relates to a plant protective composition comprising α-hydroxy-acid or a derivative thereof.

In accordance with the present invention, the term “plant protective composition” relates to a composition that provides protection of plants from natural stress conditions. Such stress conditions include, without being limiting, wounding and pathogenic infections, such as viruses, bacteria, fungi or insects but also heat, cold or drought.

A plant is considered to be protected by the inventive composition when the stress-induced damage of plant tissue or the amount of pathogens present in the plant is reduced to less than 50% of the damage or amount of pathogens found in plants not treated with the protective composition, more preferably less than 40%, such as for example less than 30%, even more preferably less than 20%, such as less than 10% and more preferably less than 5%. Most preferably, the stress-induced damage of plant tissue or the amount of pathogens present in the plant is reduced to zero, i.e. there is no detectable damage of tissues and no detectable amounts of pathogens present. Methods for determining the degree of plant tissue damage and for determining the amount of pathogens present are well known in the art and include, without being limiting, determination of lesion size, quantification of pathogen e.g. by quantitative PCR, determination of yield loss as well as the method described herein below in the appended examples.

The term “α-hydroxy-acid”, as used herein, relates to a class of chemical compounds that consist of a carboxylic acid substituted with a hydroxyl group on the adjacent carbon. Non-limiting common examples of α-hydroxy-acids include isoleucic acid, valic acid, glycolic acid, lactic acid and mandelic acid.

Methods for obtaining α-hydroxy-acids are well known in the art and include, without being limiting, the isolation of naturally occurring α-hydroxy-acids, for example from fruit or synthesis of α-hydroxy-acid. Methods for the synthesis of α-hydroxy-acid have been described in the art, for example in Mamer 2000, Methods in Enzymology, volume 324, pages 3 to 10; Yabuuchi and Kusumi, 1999; Caille et al., 2009 as well as in U.S. Pat. No. 4,981,619, U.S. Pat. No. 7,002,039 or US20110098438. A further reference describing the synthesis of α-hydroxy-acid is Snowden et al., 2005. α-hydroxy-acid can also be easily obtained from α-keto-acids (such as amino acid precursors) by reduction. α-hydroxy-acid can also be obtained from amino acids by reaction with acidic NaNO₂. Alternatively, α-hydroxy-acids may be obtained commercially, such as for example from Sigma-Aldrich (Germany) or Interchim (France).

Methods for obtaining derivatives of a α-hydroxy-acid are known in the art and comprise methods of modifying the α-hydroxy-acid to achieve: (i) a modified site of action and/or spectrum of activity and/or (ii) improved potency, and/or (iii) decreased side effects, and/or (iv) modified onset of action, duration of effect, and/or (v) modified metabolic parameters involving resorption, and metabolism, and/or (vi) modified physico-chemical parameters (solubility, hygroscopicity, color, odor, stability, state), and/or (vii) improved general specificity and/or (viii) optimized application form and route by (a) esterification, for example esterification of carboxyl groups, or esterification of hydroxyl groups with carboxylic acids, or esterification of hydroxyl groups to, e.g. phosphates, pyrophosphates or sulfates or hemi-succinates, or (b) formation of salts, or (c) formation of complexes, or (d) esterification by intermolecular dimerisation of the α-hydroxy-acid, or (e) introduction of hydrophilic moieties, or (f) introduction/exchange of substituents on side chains, change of substituent pattern, or (g) modification by introduction of isosteric or bioisosteric moieties, or (h) introduction of branched side chains, or (i) conversion of alkyl substituents to cyclic analogues, or (j) derivatisation of hydroxyl group to ketales, acetales, or (k) N-acetylation to amides, phenylcarbamates, or (l) modifications that prevent the inactivation via endogenous glycosylation in the plant, such as for examples formation of ethers or esters, replacement of —OH with —SH or amidation.

In accordance with the present invention, one or more α-hydroxy-acids or derivatives thereof are comprised in the composition. For example, two different α-hydroxy-acids or derivatives thereof, such as e.g. three, four, five, six, seven, eight, nine or ten different α-hydroxy-acids or derivatives thereof or more may be comprised in the plant protective composition. It will be readily understood by a person skilled in the art that the herein recited number of different α-hydroxy-acids or derivatives thereof is intended to limit the number of different types of α-hydroxy-acids or derivatives thereof, but not the number of molecules of one type thereof. Thus, for example the term “three different α-hydroxy-acids or derivatives thereof”, refers to three different types of α-hydroxy-acids or derivatives thereof, wherein the amount of each individual α-hydroxy-acid or derivative thereof is not particularly limited. Where more than one α-hydroxy-acid or derivative thereof is comprised in the composition, the number of α-hydroxy-acids or derivatives thereof can be selected independently of each other, e.g. the composition may comprise two α-hydroxy-acids and three derivatives thereof.

In accordance with the present invention, it was surprisingly found that α-hydroxy acids activate plant defence pathways and can be modulated by endogenous small-molecule glucosylation. Importantly, neither isoleucic acid nor its glucoside had been described before in plants.

Isoleucic acid was identified as the substrate of the Arabidopsis thaliana glucosyltransferase isoform UGT76B1. The UGT76B1 gene was found in a scan of public expression data for stress-responsiveness among UGT genes as the top stress-inducible member of this family. UGT76B1 was broadly up-regulated by both abiotic and biotic cues. UGT76B1 is present as a single isoform in its subclass in Arabidopsis thaliana. Analysis of related Brassicaceae genomes revealed a highly conserved, single copy homolog.

Despite major advances in plant biology due to genome annotations and omics approaches, a majority of gene products are still orphan enzymes without specific substrates and physiological roles (Fridman and Pichersky, 2005; Saito et al., 2008; Hanson et al., 2010).

Although the annotation of an encoded enzyme e.g. as a UGT most probably denotes its activity as a transferase of an activated sugar onto small molecule acceptors, this knowledge does not provide a clue towards its native substrate(s), not to mention its in vivo function. In the case of UGTs, even sequence homology to already known isoforms does not allow to deduce substrate classes (Vogt and Jones, 2000; Bowles et al., 2006). Nevertheless, integration of metabolite profiling with independent evidence, in particular of transcriptional co-expression and comparative genomics, has strongly assisted the elucidation of metabolic pathways and assignment of enzymatic activities (Hirai et al., 2005; Yonekura-Sakakibara et al., 2008; Matsuda et al., 2009; Ohta et al., 2010). In the case of the broadly stress-inducible UGT76B1 gene however, co-expression analyses did not indicate an assignment, which could hint towards a class of potential substrates. Thus, a non-targeted approach employing ultra-high resolution FT-ICR mass spectrometry was employed in order to obtain information on the affected pathway or substrate without any other prior knowledge. Enzymatic tests using the recombinant enzyme confirmed its ability to glucosylate the predicted aglycon in vitro and thereby established the α-hydroxy-acid isoleucic acid as the UGT76B1 substrate.

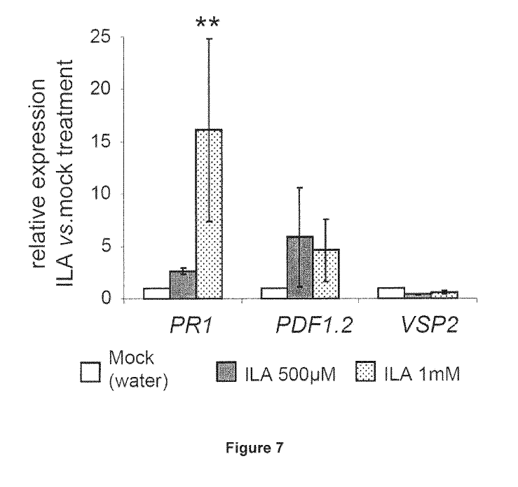

As is shown in the appended examples, exogenously applied isoleucic acid promoted the SA pathway and resulted in the upregulation of the SA-response gene PR1 (see FIG. 7, Example 9).

In a preferred embodiment of the plant protective composition of the invention, the α-hydroxy-acid is represented by the general formula (I):

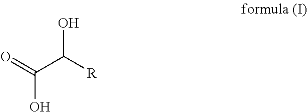

wherein R is hydrogen or a linear or branched C1 to C6 alkyl group.

In accordance with this embodiment, preferred α-hydroxy-acids are α-hydroxy-acids having two to eight carbon atoms and which contain a linear or branched alkyl moiety. In accordance with the present invention, the number of carbon atoms indicated by the name of an α-hydroxy acid includes those carbon atoms which may be contained in methyl or ethyl branches present on the main chain of the alkyl moiety, unless those branches are specifically and additionally recited (e.g. the term “pentanoic acid” includes both α-hydroxy-acids with a five linear carbon atoms as well as e.g. butyric acid with a methyl branch while the term “3-methyl-pentanoic acid” refers to an α-hydroxy-acid with five linear carbon atoms plus a methyl branch in position 3, thus resulting in a total of six carbon atoms). Accordingly, the α-hydroxy acid may be referred to as 2-hydroxy-ethanoic acid (glycolic acid), 2-hydroxypropanoic acid (lactic acid), 2-hydroxy-butyric acid, 2-hydroxy-pentanoic acid, 2-hydroxy-hexanoic acid, 2-hydroxy-heptanoic acid and 2-hydroxy-octanoic acid.

Most preferred among the compounds of general formula I are those compounds wherein R is H, or a linear C1 to C6 alkyl group, or a branched C2 to C6 alkyl group which provides a methyl substituent in β-position (which are also referred to as position 3 herein) relative to the carboxylic acid group, or a branched C3 to C6 alkyl group which provides a methyl substituent in γ-position (which are also referred to as position 4 herein) relative to the carboxylic acid group.

In accordance with the present invention, the term “α-hydroxy-acid” or the recited preferred α-hydroxy-acids include all isomeric forms of the respective α-hydroxy-acids.

Thus, the term “2-hydroxy propanoic acid” (lactic acid) includes α-hydroxy-acids of the general formula C₃H₆O₃, such as (S)-2-hydroxy propanoic acid or (R)-2-hydroxy propanoic acid.

The term “2-hydroxy-butyric acid” includes α-hydroxy-acids of the general formula C₄H₈O₃, such as (S)-2-hydroxy-butyric acid or (R)-2-hydroxy-butyric acid.

The term “2-hydroxy-pentanoic acid” includes α-hydroxy-acids of the general formula C₅H₁₀O₃, such as e.g. (S)-2-hydroxy-pentanoic acid or (R)-2-hydroxy-pentanoic acid; 2-hydroxy-3-methyl-butyric acid (valic acid, 2-hydroxyisovaleric acid), such as 2S-hydroxy-3-methyl-butyric acid or 2R-hydroxy-3-methyl-butyric acid.

Furthermore, the term “2-hydroxy-hexanoic acid” includes α-hydroxy-acids of the general formula C₆H₁₂O₃, such as e.g. 2S-hydroxy-hexanoic acid and 2R-hydroxy-hexanoic acid; 2-hydroxy-3-methyl-pentanoic acid (isoleucic acid, 2-Hydroxy-3-methylvaleric acid) such as 2S,3S-2-hydroxy-3-methyl-pentanoic acid, 2S,3R-2-hydroxy-3-methyl-pentanoic acid, 2R,3S-2-hydroxy-3-methyl-pentanoic acid or 2R,3R-2-hydroxy-3-methyl-pentanoic acid; 2-hydroxy-4-methyl-pentanoic acid, such as 2S-hydroxy-4-methyl-pentanoic acid, 2R-hydroxy-4-methyl-pentanoic acid; 2-hydroxy-3,3-dimethyl-pentanoic acid such as 2S-hydroxy-3,3-dimethyl-pentanoic acid, 2R-hydroxy-3,3-dimethyl-pentanoic acid.

The term “2-hydroxy-heptanoic acid” includes α-hydroxy-acids of the general formula C₇H₁₄O₃, such as e.g 2S-hydroxy-heptanoic acid and 2R-hydroxy-heptanoic acid; 2-hydroxy-3-methyl-hexanoic acid such as 2S-hydroxy-3S-methyl-hexanoic acid, 2S-hydroxy-3R-methyl-hexanoic acid, 2R-hydroxy-3S-methyl-hexanoic acid, 2R-hydroxy-3R-methyl-hexanoic acid; 2-hydroxy-4-methyl-hexanoic acid such as 2S-hydroxy-4S-methyl-hexanoic acid, 2S-hydroxy-4R-methyl-hexanoic acid, 2R-hydroxy-4S-methyl-hexanoic acid, 2R-hydroxy-4R-methyl-hexanoic acid; 2-hydroxy-5-methyl-hexanoic acid such as 2S-hydroxy-5-methyl-hexanoic acid and 2R-hydroxy-5-methyl-hexanoic acid; 2-hydroxy-3,3-dimethyl-pentanoic acid such as 2S-hydroxy-3,3-dimethyl-pentanoic acid, 2R-hydroxy-3,3-dimethyl-pentanoic acid; 2-hydroxy-3,4-dimethyl-pentanoic acid such as 2S-hydroxy-3S,4-dimethyl-pentanoic acid, 2S-hydroxy-3R,4-dimethyl-pentanoic acid, 2R-hydroxy-3S,4-dimethyl-pentanoic acid, 2R-hydroxy-3R,4-dimethyl-pentanoic acid; 2-hydroxy-4,4-dimethyl-pentanoic acid such as 2S-hydroxy-4,4-dimethyl-pentanoic acid, 2R-hydroxy-4,4-dimethyl-pentanoic acid.

The term “2-hydroxy-octanoic acid” includes α-hydroxy-acids of the general formula C₈H₁₆O₃, such as e.g 2S-hydroxy-octanoic acid, 2R-hydroxy-octanoic acid; 2-hydroxy-3-methyl-heptanoic acid such as 2S-hydroxy-3S-methyl-heptanoic acid, 2S-hydroxy-3R-methyl-heptanoic acid, 2R-hydroxy-3S-methyl-heptanoic acid, 2R-hydroxy-3R-methyl-heptanoic acid; 2-hydroxy-4-methyl-heptanoic acid such as 2S-hydroxy-4S-methyl-heptanoic acid, 2S-hydroxy-4R-methyl-heptanoic acid, 2R-hydroxy-4S-methyl-heptanoic acid, 2R-hydroxy-4R-methyl-heptanoic acid; 2-hydroxy-5-methyl-heptanoic acid such as 2S-hydroxy-5S-methyl-heptanoic acid and 2S-hydroxy-5R-methyl-heptanoic acid, 2R-hydroxy-5S-methyl-heptanoic acid, 2R-hydroxy-5R-methyl-heptanoic acid; 2-hydroxy-6-methyl-heptanoic acid such as 2S-hydroxy-6-methyl-heptanoic acid, 2R-hydroxy-6-methyl-heptanoic acid; 2-hydroxy-3,4-dimethyl-hexanoic acid such as 2S-hydroxy-3S,4S-dimethyl-hexanoic acid, 2S-hydroxy-3S,4R-dimethyl-hexanoic acid, 2S-hydroxy-3R,4S-dimethyl-hexanoic acid, 2R-hydroxy-3S,4S-dimethyl-hexanoic acid, 2S-hydroxy-3R,4R-dimethyl-hexanoic acid, 2R-hydroxy-3R,4R-dimethyl-hexanoic acid, 2R-hydroxy-3R,4S-dimethyl-hexanoic acid, 2R-hydroxy-3S,4R-dimethyl-hexanoic acid; 2-hydroxy-3,5-dimethyl-hexanoic acid such as 2S-hydroxy-3S,5-dimethyl-hexanoic acid, 2S-hydroxy-3R,5-dimethyl-hexanoic acid, 2R-hydroxy-3S,5-dimethyl-hexanoic acid, 2R-hydroxy-3R,5-dimethyl-hexanoic acid; 2-hydroxy-4,5-dimethyl-hexanoic acid such as 2S-hydroxy-4S,5-dimethyl-hexanoic acid, 2S-hydroxy-4R,5-dimethyl-hexanoic acid, 2R-hydroxy-4S,5-dimethyl-hexanoic acid, 2R-hydroxy-4R,5-dimethyl-hexanoic acid; 2-hydroxy-3,3-dimethyl-hexanoic acid such as 2S-hydroxy-3,3-dimethyl-hexanoic acid, 2R-hydroxy-3,3-dimethyl-hexanoic acid; 2-hydroxy-4,4-dimethyl-hexanoic acid such as 2S-hydroxy-4,4-dimethyl-hexanoic acid, 2R-hydroxy-4,4-dimethyl-hexanoic acid; 2-hydroxy-5,5-dimethyl-hexanoic acid such as 2S-hydroxy-5,5-dimethyl-hexanoic acid, 2R-hydroxy-5,5-dimethyl-hexanoic acid.

It will be appreciated by the skilled person that the reference to a particular α-hydroxy-acid, such as e.g. 2-hydroxy-pentanoic acid, includes all possible isomers falling under said term as well as specific isomers in purified form (i.e. separated from other isomeric forms of the same formula). In other words, where the plant protective composition comprises such an α-hydroxy-acid, it may comprise a mixture of different isomers or it may comprise one specific isomeric form of said α-hydroxy-acid. It will further be appreciated by the skilled person that not all isomers of a particular α-hydroxy-acid necessarily have the same activity. It is within the skills of said skilled person to choose the isomer or isomer mixture most suitable for his needs.

Most preferably, the α-hydroxy-acid is 2-hydroxy-3-methyl-pentanoic acid (isoleucic acid). Even more preferably, the α-hydroxy-acid is 2S,3S-2-hydroxy-3-methyl-pentanoic acid.

In another preferred embodiment of the plant protective composition of the present invention, the derivative is selected from the group consisting of an ester or an anhydride of an α-hydroxy-acid.

Esters of α-hydroxy-acids comprise compounds wherein the hydrogen in the carboxylic acid group is replaced by a hydrocarbon group, such as e.g. an alkyl group like methyl or ethyl or octyl, or an aryl-containing group like phenyl. The ester can also be an intermolecular ester.

Anhydrides of α-hydroxy-acids comprise compounds wherein two α-hydroxy-acids are linked via their carboxylic acid group forming an anhydride (e.g. R₁—C(O)—O—C(O)—R₂), thus resulting in dimeric α-hydroxy-acids.

In another preferred embodiment of the plant protective composition of the invention, the composition further comprises a carrier and/or additive.

Suitable carriers and additives are well known in the art and may be solid, semisolid or liquid compounds. Non-limiting examples of carriers include fillers, diluents, encapsulating material or formulation auxiliary of any type such as e.g. solvents, natural or regenerated mineral substances, thickeners, binders, pH adjusting compounds. Non-limiting examples of additives comprise tackifiers, emulsifiers, dispersants, wetting agents, micronutrient donors, fertilisers or other preparations that influence plant growth.

Preferred examples of solvents include aromatic hydrocarbons, preferably the fractions containing 8 to 12 carbon atoms, e.g. xylene mixtures or substituted naphthalenes, phthalates such as dibutyl phthalate or dioctyl phthalate, aliphatic hydrocarbons such as cyclohexane or paraffins, alcohols and glycols and their ethers and esters, such as ethanol, ethylene glycol, ethylene glycol monomethyl or monoethyl ether, ketones such as cyclohexanone, strongly polar solvents such as N-methyl-2-pyrrolidone, dimethyl sulfoxide or dimethylformamide, as well as vegetable oils or epoxidized vegetable oils, such as epoxidized coconut oil or soybean oil; or water.

For dusts and dispersible powders, solid carriers are generally employed. Such solid carriers may be selected from e.g. natural mineral fillers such as calcite, talcum, kaolin, montmorillonite or attapulgite. Highly dispersed silicic acid or highly dispersed absorbent polymers may be added in order to improve the physical properties. Non-limiting examples for granulated adsorptive carriers are carriers of a porous type, for example pumice, sepiolite or bentonite; while non-limiting examples of non-adsorbent carriers include calcite or sand. Furthermore, pre-granulated materials of inorganic or organic nature can be used, e.g. dolomite or pulverised plant residues.

Examples of advantageous application-promoting additives also include e.g. natural or synthetic phospholipids of the series of the cephalins and lecithins.

Properties such as emulsifying, dispersing and wetting are influenced by the addition of surface-active compounds, or mixtures thereof, including non-ionic, cationic and/or anionic surfactants. Non-ionic surfactants include, without being limiting, polyglycol ether derivatives of aliphatic or cycloaliphatic alcohols, saturated or unsaturated fatty acids and alkylphenols, said derivatives containing 3 to 30 glycol ether groups and 8 to 20 carbon atoms in the (aliphatic) hydrocarbon moiety and 6 to 18 carbon atoms in the alkyl moiety of the alkylphenols. Non-limiting examples of cationic surfactants include quaternary ammonium salts which contain, as N-substituent, at least one C₈-C₂₂ alkyl radical and, as further substituents, un-substituted or halogenated lower alkyl, benzyl or hydroxy-lower alkyl radicals. Anionic surfactants can be selected from water-soluble soaps and water-soluble synthetic surface-active compounds. Suitable soaps are alkali metal salts, alkaline earth metal salts or un-substituted or substituted ammonium salts of higher fatty acids (C₁₀-C₂₂), e.g. the sodium or potassium salts of oleic or stearic acid or of natural fatty acid mixtures which can be obtained e.g. from coconut oil or tallow oil. Synthetic surfactants include, without being limiting, fatty alcohol sulfonates, fatty alcohol sulfates, sulfonated benzimidazole derivatives or alkylsulfonates.

Further additives may be selected from the group of binders, penetration enhancers, such as e.g. detergents, stabilizers, agents improving the odor of the composition, antifoaming agents, viscosity regulators, pH regulators and pH stabilizers.

The plant protective composition of the invention may be prepared by homogeneously mixing and/or grinding the active ingredient(s) together with the remaining ingredients, such as e.g. the solid or liquid carrier or additive.

In a further preferred embodiment of the plant protective composition of the invention, the composition is selected from the group consisting of directly sprayable or dilutable solutions, aqueous solutions, emulsifiable concentrates, coatable pastes, dilute emulsions, wettable powders, soluble powders, dusts, granulates, encapsulations in e.g. polymeric substances and natural or synthetic substances impregnated with the active compound.

Furthermore, the plant protective composition may also comprise additional active agents with fungicidal, bactericidal or virucidal activity or other compounds suitable to activate the plants' own defense system. Such compounds are well known in the art and examples for the first type of compound include, without being limiting, insecticides, fungicides, bactericides, nematicides, herbicides, molluscicides while examples for the second type of compound include, without being limiting, the chloronicotinyl or benzothiadiazole-derivates described herein above (e.g. US 2009/0018019 or U.S. Pat. No. 4,931,581) or mixtures of several of these active agents. Further examples of commonly employed active agents suitable for combination with the plant protective composition of the present invention include, without being limiting, tebuconazol, fludioxonil, metconazol, thiophanat-methyl, fluoxastrobin, prothioconazol, prochloraz, fluquinconazol, spiroxamine, difenoconazol, epoxiconazol, prothioconazol, triticonazol, dimoxystrobin, dimethoat, lambda-cyhalothrin, thiamethoxam, pirimiphos-methyl, metaflumizone, thiacloprid, beta-cyfluthrin, imidacloprid, spinosad, chlorantraniliprole, clothianidin, deltamethrin, diflubenzuron, spirodiclofen, alpha-cypermethrin, zeta-cypermethrin, boscalid, dimoxystrobin, metconazol, mepiquat or triadimenol.

In another preferred embodiment of the plant protective composition of the invention, the concentration of the α-hydroxy-acid or derivative thereof in the composition is between 1 μM and 2 mM.

More preferably, the concentration of the α-hydroxy-acid or derivative thereof in the composition is between 50 μM and 1 mM, more preferably between 100 μM and 800 μM and most preferably between 200 μM and 500 μM. Any numerical values not explicitly mentioned above but falling within the above recited preferred ranges are also envisaged herein. Alternatively, the application rate may be expressed as the amount of active ingredient per hectare to be treated. Preferably, the application rate is from 50 g to 5 kg of the α-hydroxy-acid or derivative thereof per hectare, more preferably from 100 g to 2 kg of the α-hydroxy-acid or derivative thereof per hectare and most preferably from 150 g to 600 g of the α-hydroxy-acid or derivative thereof per hectare.

In another preferred embodiment of the plant protective composition of the invention, treatment with the composition reduces the stress-induced damage of plant tissue or the amount of pathogens present in the plant to less than 50% of the damage or amount of pathogens found in plants not treated with the protective composition. As defined herein above, treatment with the composition more preferably reduces the stress-induced damage of plant tissue or the amount of pathogens present in the plant to less than 40%, such as for example less than 30%, even more preferably less than 20%, such as less than 10% and more preferably less than 5%. Most preferably, the stress-induced damage of plant tissue or the amount of pathogens present in the plant is reduced to zero, i.e. there is no detectable damage of tissues and no detectable amounts of pathogens present.

In a further preferred embodiment of the plant protective composition of the invention, the composition protects the plant against pathogens.

In a more preferred embodiment of the plant protective composition of the invention, the pathogens are selected from the group consisting of bacteria, fungi and viruses.

More preferably, the pathogens are selected from the group consisting of Pseudomonas, such as e.g. P. syringae, P. aeruginosa, P. chlororaphis, P. fluorescens, P. pertucinogena, P. putida or P. stutzeri; Hyaloperonospora, such as e.g. H. arabidopsidis, H. brassicae or H. parasitica; oomycetes, such as e.g. Plasmopara viticola, Phytophthora nicotianae, Peronospora tabacinae or Phytophthora infestans, basidiomycetes, such as e.g. the genera Hemileia, Rhizoctonia, Puccinia or Phakopsora; ascomycets, such as e.g. Venturia inaequalis, Ramularia gossypii, Sphaerotheca fuliginea, Sclerotinia homoeocarpa, Colletotrichum graminicola, Erysiphe, Monilinia, Uncinula or the genera Curvularia Pyrenophora; xanthomonads, such as for example X. oryzae or X. vesicatoria; Erwinia (for example E. amylovora); fungi imperfecti, such as for example Colletotrichum lagenarium, Piricularia oryzae or Cercospora nicotinae, and viruses, such as e.g. the mosaic viruses such as tobacco or barley, potato leaf roll virus.

In accordance with the present invention, independent ugt76b1 knockout lines, which cannot glucosylate isoleucic acid, exhibited enhanced resistance towards Pseudomonas syringae infections. This is accompanied by constitutively elevated SA levels and SA-related marker gene expression, whereas JA-dependent marker genes are repressed. Conversely, UGT76B1 over-expression causes the opposite reactions, as it attenuates SA-dependent plant defense in the absence of infection and promotes JA response. Furthermore, as shown herein, exogenously applied isoleucic also promoted the SA pathway and resulted in the upregulation of the SA-response gene PR1.

In another preferred embodiment of the plant protective composition of the invention, the composition induces an endogenous plant resistance mechanism.

It was found in accordance with the present invention that the inventive composition induces the expression of PR proteins, which are known in the art to be SA-responsive genes that are involved in defense responses of plants. Thus, without wishing to be bound by any theory, it is suggested that the composition of the present invention does not have a direct action against the pathogens themselves but, instead, the composition protects plants by activating the plants' own defence mechanisms. Preferably, the plants' own biological defence system is activated and stimulated before the plant is stressed, i.e. the plant protective composition of the invention is applied to plants in order to prevent the negative effects of the various stress factors.

In another preferred embodiment of the plant protective composition of the present invention, the plant is selected from the group consisting of monocotyledonous plants and dicotyledonous plants.

The term “monocotyledonous plants” refers to a group of plants that is characterized by having one seed-leaf (cotyledon), while the term “dicotyledonous plants” refers a second group of plants characterized by having two embryonic leaves. Non-limiting examples of monocotyledonous plants include wheat, oats, millet, barley, rye, maize, rice, sorghum, triticale, spelt and sugar cane while non-limiting examples of dicotyledonous plants include Arabidopsis, fibre plants (cotton, flax, hemp, jute), buckwheat, vines, tea, hops, pistachio, cress, linseed, oil plants (rape, mustard, poppy, olives, sunflowers, coconut, castor oil plants, cocoa beans, groundnuts), vegetables (e.g. spinach, lettuce, asparagus, cabbages, carrots, onions, tomatoes, potatoes, paprika, brassicas), aubergines, corn, tobacco, tagetes, calendula, cucumber plants (such as cucumber, marrows, melons), soft fruit (e.g. apples, pears, plums, peaches, almonds, cherries, strawberries, raspberries and blackberries), citrus fruit (such as oranges, lemons, grapefruit, mandarins), pumpkin/squash, courgette, beet (e.g. sugar beet and fodder beet), drupes (e.g. coffee, jujube, mango, palms such as e.g. date palms), lauraceae (e.g. avocados, cinnamon, camphor), ornamentals (e.g. flowers, shrubs, deciduous trees and conifers) and legumes (such as beans, lentils, peas, soybeans).

Preferably, the plant is selected from Arabidopsis, cucumber, tobacco, vine, rice, cereals (such as wheat), pear, pepper, potato, tomato and apple. Also preferred is that the plant is selected from Arabidopsis, cucumber, tobacco, vine, rice, cereals (such as wheat, barley), pear, pepper, potato, tomato and apple.

In accordance with the present invention, the plants can be traditional crop plants or plant varieties having new properties, which have been obtained by breeding with conventional methods, mutagenesis or by recombinant DNA techniques. Thus, the plants may include transgenic plants and plant hybrids.

The present invention also relates to a method of protecting plants from pathogens comprising contacting the plants with the plant protective composition of the invention.

In accordance with the present invention, the plants can be contacted with the plant protective composition of the invention by any method known in the art. In a preferred embodiment of the method of the invention, the plants are contacted by any one selected from the group consisting of spraying, scattering, pouring, coating and dusting.

Accordingly, a spray may be provided comprising the plant protective composition of the invention in liquid form dispersed in a gas, such that small droplets of the composition are formed. The spray then enables to distribute the compositions over a surface area, such as for example a single plant or a field comprising a plurality of plants. The dispersion of the composition in a gas is also referred to as atomizing. The composition in a liquid state may also be scattered onto plants or a field or may be poured onto the plants or a field. Furthermore, parts of the plant or entire plants can be coated with the compositions of the present invention, for example by dipping the plant into the composition or by brushing the plants, or parts thereof, with the composition.

Alternatively, or additionally, the composition may be applied by dusting, i.e. the (aerial) application of the composition in powder form.

Furthermore, the composition may also be introduced into the soil on which the plants are growing, for example in form of a liquid, granules, pellets or a stick, which can e.g. disintegrate with time in order to release the composition of the invention.

The above described means of application lead to either a foliar application, application to the stem or buds, application to (and uptake through) the roots in case of application to the soil or application to the seeds.

It will be appreciated that the particular method of application has to be selected depending on the respective circumstances and the target of the treatment.

All the definitions and preferred embodiments described herein with regard to the plant protective composition apply mutatis mutandis also to the method of the invention. For example, the preferred pathogens, additional ingredients defined with regard to the composition also apply to the method of the invention. Furthermore, the method of the present invention may comprise the additional treatment, either simultaneously or subsequently, with active agents having bactericidal, virucidal or fungicidal activity or stimulating the plants' own defense systems, as described herein above with regard to the inventive plant protective composition.

Unless otherwise defined, all technical and scientific terms used herein have the same meaning as commonly understood by one of ordinary skill in the art to which this invention belongs. In case of conflict, the patent specification, including definitions, will prevail.

The figures show:

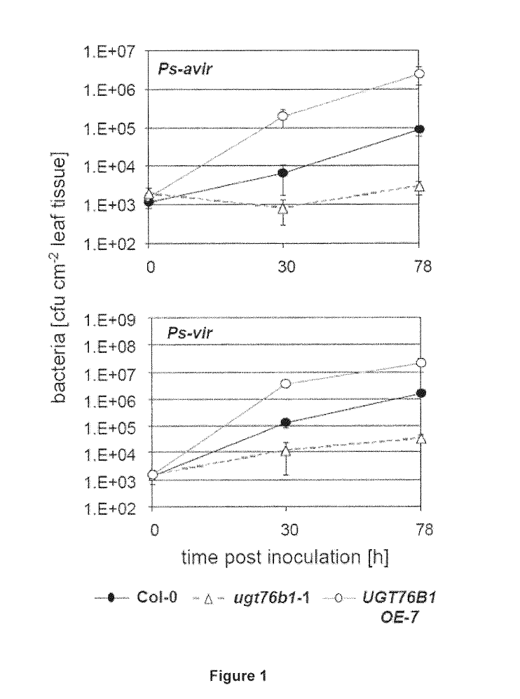

FIG. 1. Bacterial growth of avirulent and virulent Pseudomonas syringae in Arabidopsis leaves of wild-type, ugt76b1-1 (knockout) and UGT76B1-OE-7 (constitutive overexpression) plants.

Leaves were infiltrated with an inoculum of 5*10⁵ cfu mL⁻¹ of Ps-avir (upper graph) and Ps-vir (lower graph). Bacteria (cfu cm⁻²) were quantified 30 h and 78 h after inoculation. The graphs represent the means and standard deviations of three replicates. The experiment was repeated with similar result.

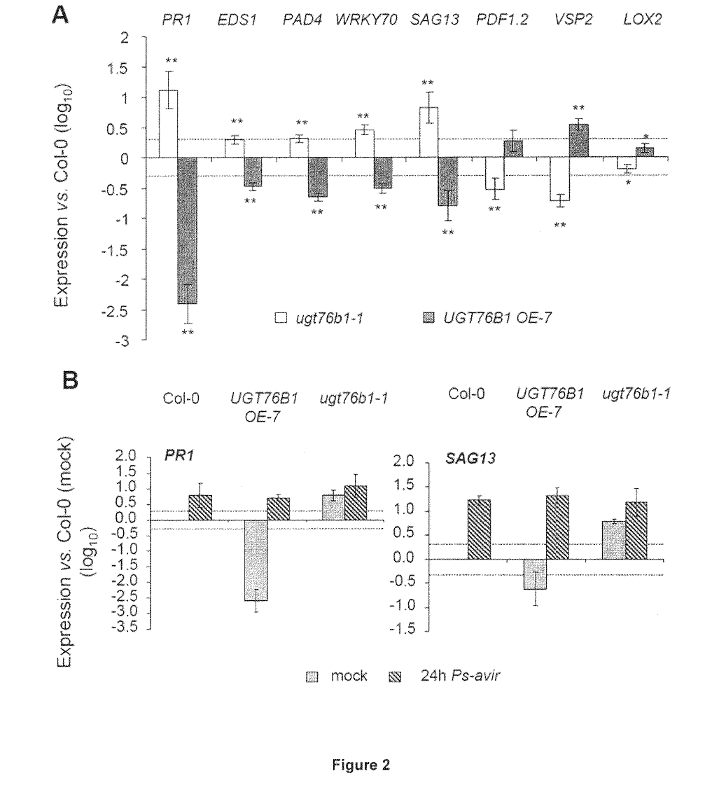

FIG. 2. Defense marker gene expression in ugt76b1-1 (knockout) and UGT76B1-OE-7 (constitutive overexpression) plants before and after pathogen infection.

(A) Gene expression of PR1, EDS1, PAD4, WRKY70, SAG13, PDF1.2, VSP2 and LOX2 in 5-week-old ugt76b1-1 and UGT76B1-OE-7 measured by RTqPCR. Expression levels are normalized to UBIQUITIN5 and S16 transcripts; levels relative to Col-0 plants are displayed. Arithmetic means and standard errors from log₁₀-transformed data of two experiments each consisting of three independent replicates were calculated using ANOVA. Stars indicate significance of the difference to the wild-type line: ** p-value<0.01, * p-value<0.05. (B) Transcript levels of PR1 and SAG13 in 5-week-old wild-type plants 30 h after infection (5*10⁵ cfu mL⁻¹ Ps-avir) measured by RTqPCR. Values are relative to expression 30 h after mock treatment and log₁₀-transformed. Bars represent the mean and standard deviation of three replicates. The dashed, horizontal lines indicate twofold change.

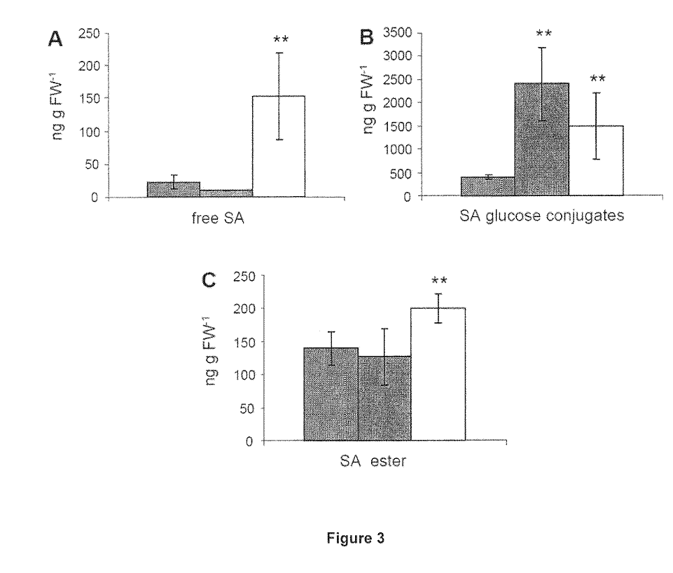

FIG. 3. Salicylic acid (SA) and conjugated SA levels in five-week-old seedlings of the wild type, ugt76b1-1 (knockout) and UGT76B1-OE-7 (constitutive overexpression).

Values represent the means and standard deviations obtained from five replicates. Stars indicate significance of the difference to the wild-type line: ** p-value<0.01. The experiment was repeated with similar results.

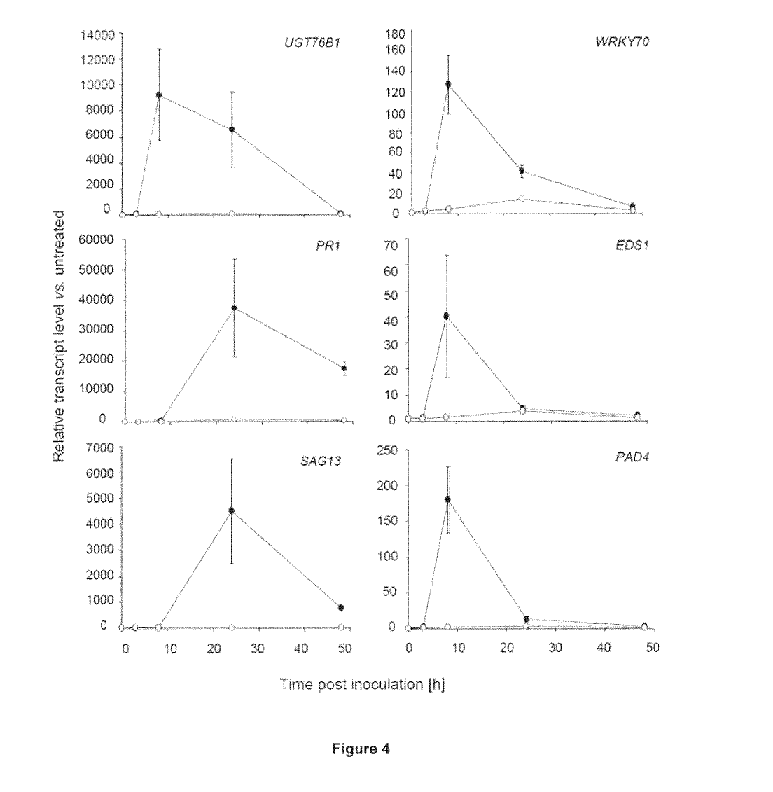

FIG. 4. RT-qPCR expression profiles of UGT76B1, WRKY70, EDS1 and PAD4 induction after infection with avirulent Pseudomonas syringae.

Transcript levels were quantified at the indicated time points after inoculation with Ps avir (closed circles) and mock (10 mM MgCl₂; open circles) treatment. The transcript level (relative expression) was normalized to the transcript abundance of UBIQUITIN5 and S16 genes. Values correspond to the mean and standard deviation of triplicates. The experiment was repeated with similar results.

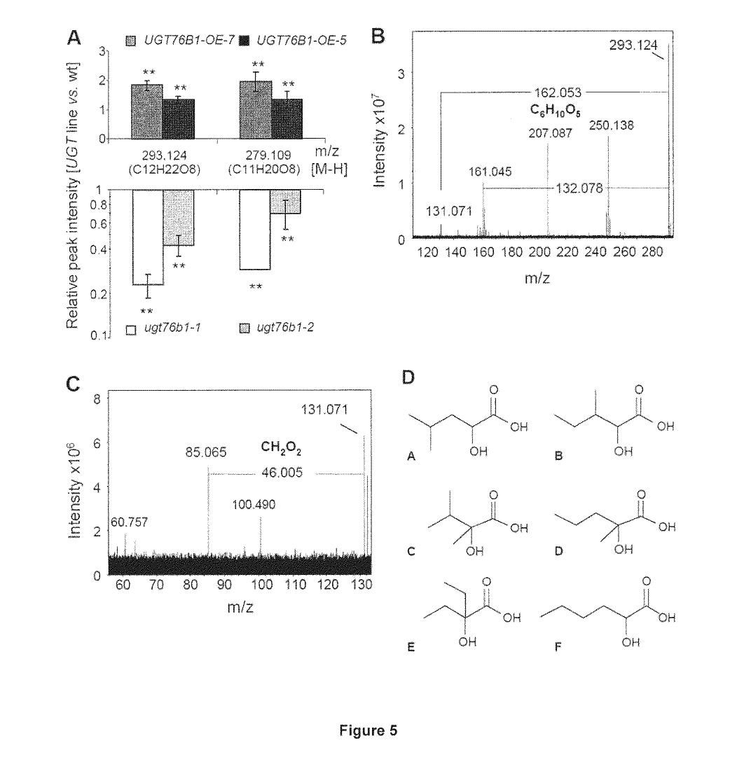

FIG. 5. Non-targeted metabolome analysis of UGT76B1 overexpression and ugt76b1 knockout lines.

(A) Metabolic changes found in roots of two independent knockout lines and two independent overexpression lines compared to the respective wild type. Means and standard deviation of three independent biological replicates with two technical replicates each are displayed. m/z 279 was nearly undetectable and undetectable in ugt76b1-2 and ugt76b1-1, respectively. Therefore, a default value for the ugt76b1-1 peak was used for calculating the relative intensity (Methods). Stars indicate significance of the difference to the wild type: ** p-value<0.01. The predicted molecular formula are indicated. The experiment was independently repeated with similar results. (B) Fragmentation pattern of m/z 293. The loss of m/z 162 confirmed the presence of a glucosidic moiety. Other major peaks at m/z 207 and 250 could be unequivocally excluded as m/z 293-derived fragments; they were originating from electrical noise and from an N-containing contaminant, respectively. In contrast, m/z 161 was in agreement with a radical anion of deprotonated glucose, which was directly produced from m/z 293. (C) Further in-cell fragmentation lead to the elimination of CH₂O₂ (formic acid), which restricted the nature of the aglycon to α-hydroxy carboxylic acid isomers. (D) Six possible isomeric molecular structures of the aglycon C₆H₁₂O₃.

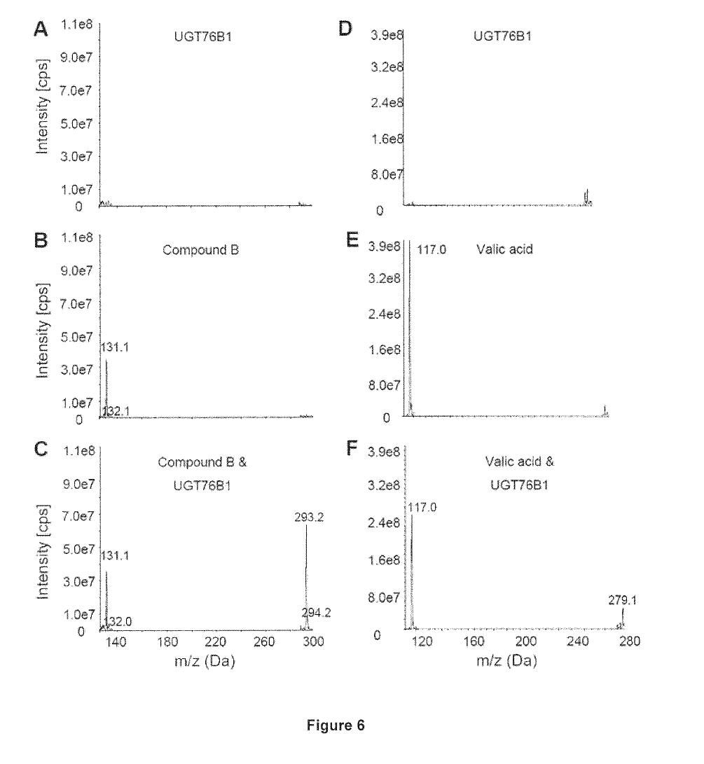

FIG. 6. In vitro activity assay of UGT76B1.

Activity of recombinant UGT76B1 was tested towards (A-C) isoleucic acid (2-hydroxy-3-methylpentanoic acid, compound B) and (D-F) valic acid. The reactions were analyzed by mass spectrometry (Methods). The m/z values of the corresponding substrates and products are indicated. The experiment was independently repeated with similar results. (A, D) Mass spectra of enzyme reactions without substrate. (B, E) Mass spectra of enzyme reactions without enzyme. (C, F) Mass spectra of complete reactions.

FIG. 7. Direct effect of exogenously applied ILA on defence marker genes.

Defense marker gene expression in Col-0 plants after isoleucic acid treatment. Transcript levels of PR1, PDF1.2 and VSP in leaves of four-week-old plants 24 h after isoleucic acid or water treatment measured by RTqPCR. Values are relative to expression 24 h after mock (water) treatment. Graph represents the mean and SD of three biological replicates. ** p-value<0.01.

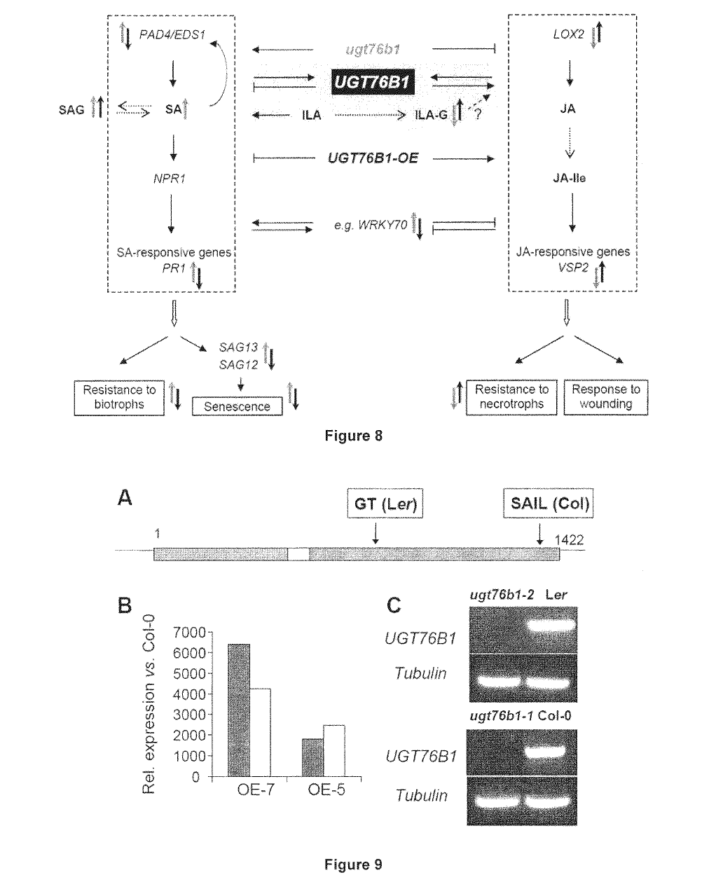

FIG. 8. Proposed model of the involvement of UGT76B1 as a novel mediator in SA- and JA-dependent regulation of defense responses and senescence.

The model relates UGT76B1 to SA and JA pathways regulating defense against (hemi-) biotrophic and necrotrophic pathogens (depicted by key steps). UGT76B1 induces the JA response and represses the SA-dependent pathway stimulating defense against necrotrophs and having a negative influence on the resistance to P. syringae and the onset of senescence. The UGT76B1 substrate isoleucic acid (ILA) enhances the SA pathway. The consequences of gain and loss of UGT76B1 function are integrated along with their dependence on SID2 and JAR1. Signaling molecules (bold), enzymatic transformations (pointed and open arrowhead), activation (closed arrowhead), suppression (^{⊥}) and important genes are indicated. Positive or negative influences by ugt76b1 (grey) and UGT76B1-OE (black) are shown.

FIG. 9. Molecular characterization of ugt76b1 knockout and UGT76B1 overexpression lines.

(A) Position of the insertions within UGT76B1 (At3g11340). (B) RTqPCR of UGT76B1 overexpression lines in two subsequent generations. Line UGT76B1-OE-7 is based on the binary vector pB2GW7, whereas pAlligator2 was used for generating UGT76B1-OE-5 (Karimi et al., 2002; Bensmihen et al., 2004). Grey bars and white bars indicate transcript levels in T2 and T3, respectively. Plant material of the T3 generation was used for subsequent experimental analyses. (C) RT-PCR analysis of UGT76B1 transcript levels in wild-type and ugt76b1 plants. TUBULIN9 (At4g20890) transcript levels were assessed as a control.

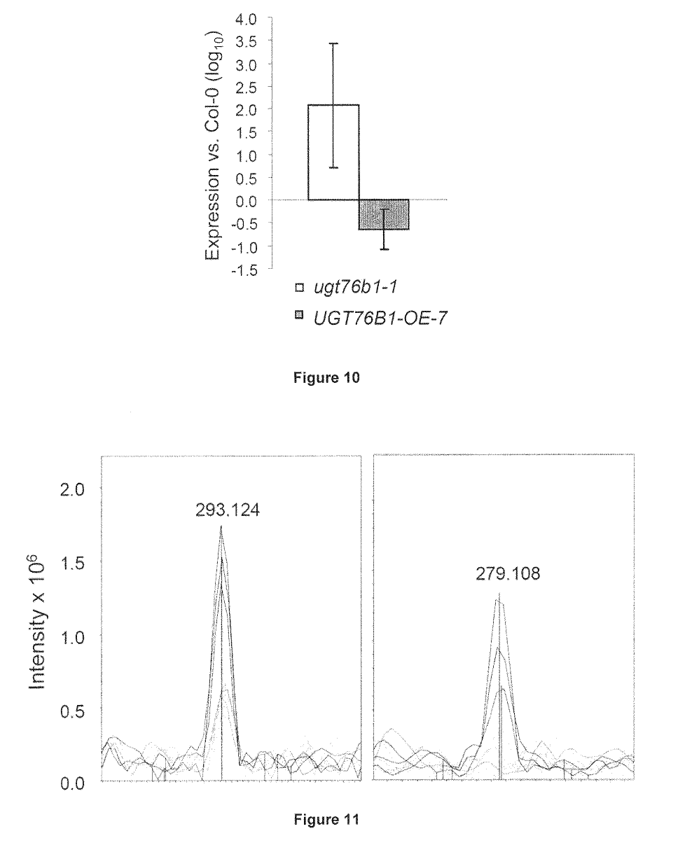

FIG. 10. Relative quantification of PR1 expression at early time point.

Graph shows relative PR1 expression in three-week-old ugt76b1-1 and UGT76B1-OE-7 plants. Expression levels were normalized to UBQ5 and S16 transcripts and expressed relative to the levels quantified for Col-0 plants (see Example 1: Methods).

FIG. 11. Detection of m/z 293.124 and m/z 279.108 in leaves of Col-0 (grey) and UGT76B1-OE-7 plants (black).

Both peaks were also significantly increased in leaf material of 4-week-old UGT76B1-OE-7 plants.

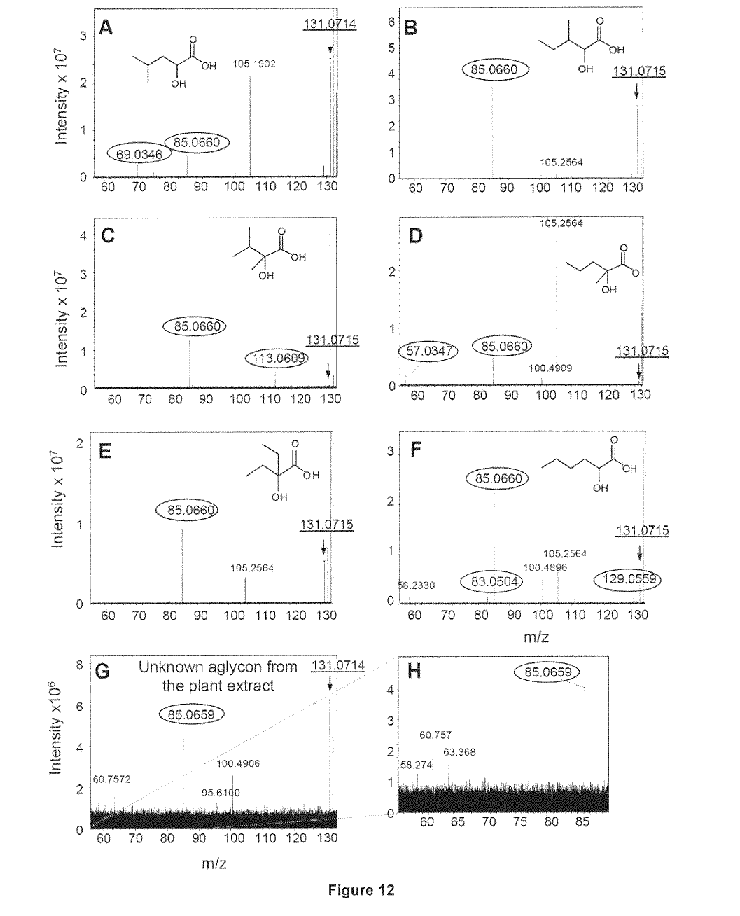

FIG. 12. Fragmentation patterns of the unknown aglycon (derived from m/z 293) from the plant extract and of putative C₆H₁₂O₃ isomers.

The precursor ion is underlined and its position indicated by an arrow. Generated fragments are encircled to distinguish them from noise peaks. The obtained fragmentation patterns of compound A and F corresponded to published data (http://www.massbank.jp/). Only structures B and E showed the same fragmentation pattern as the unknown aglycon from the plant extract (G) and were therefore selected for in vitro glucosylation studies. (A-F) Fragmentation of six C₆H₁₂O₃ isomeric reference compounds as indicated. (G, H) Fragmentation of the plant extract-derived aglycon. The region below m/z=85 is enlarged in (H) to visualize the absence of fragments observed in experiments with some of the isomeric reference compounds.

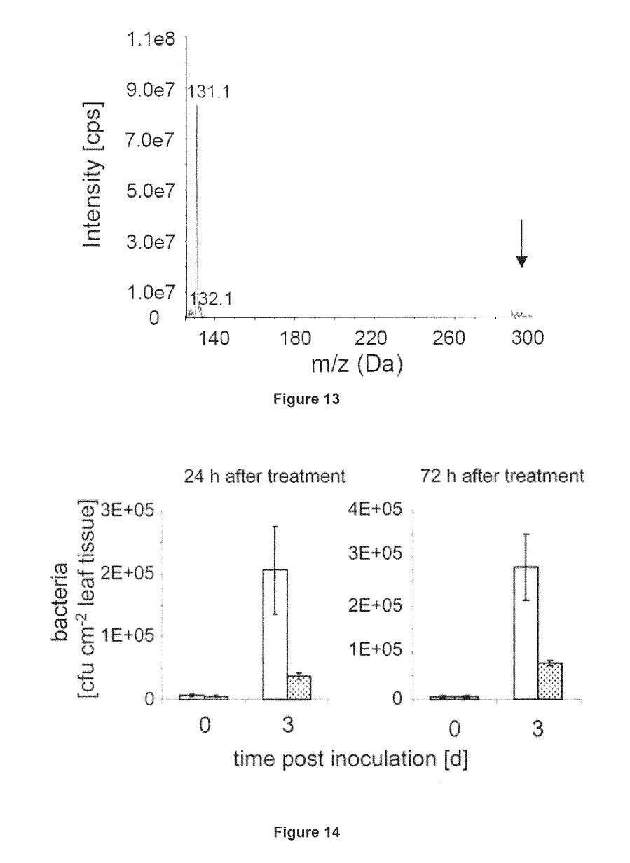

FIG. 13. In vitro activity assay of UGT76B1 towards 2-ethyl-2-hydroxybutyric acid.

Activity of recombinant UGT76B1 was tested towards 2-ethyl-2-hydroxybutyric acid (compound E) (Methods). Arrow indicates the expected mass for a potential product, which was not found here in contrast to FIG. 6C.

FIG. 14: Direct effect of exogenously applied ILA on pathogen defence. Bacterial growth in Arabidopsis leaves of wild-type plants sprayed with water (blank) or 1 mM ILA (dotted) 24 h or 72 h before infection. Plants were inoculated with 5*10⁵ cfu ml⁻¹ of Ps-avir and bacteria (cfu cm⁻²) were quantified 0 and 3 days after inoculation. The graphs represent the means and standard deviations of three replicates.

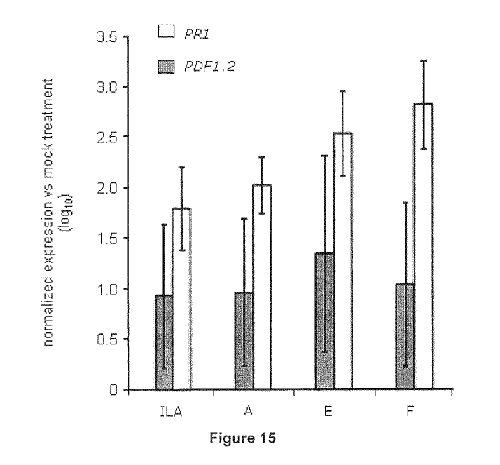

FIG. 15: Activity of additional α-hydroxy-acids as represented by induction of expression of defence marker genes PR1 and PDF1.2. Defence marker gene expression in Col-0 plants 24 h after treatment with several α-hydroxy-acids (1 mM, dissolved in water). The structure of the additional α-hydroxy-acids referred to as A, E and F is shown in FIG. 5D. Expression levels were normalized to UBQ5 and S16 transcripts and expressed relative to the levels quantified for mock treated plants (see Example 1: Methods).

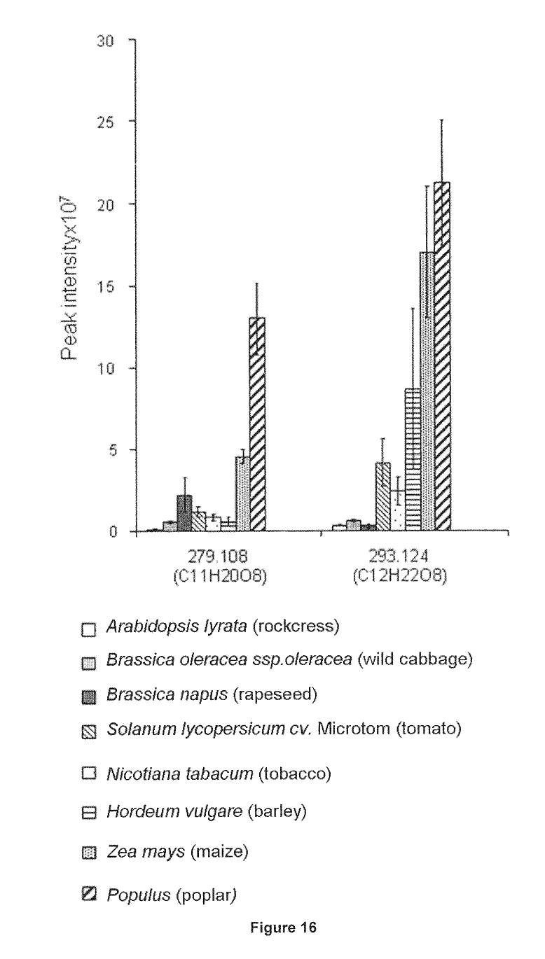

FIG. 16: Intensity of mass peaks corresponding to ILA- and valic acid-glucoside in several plant species. Methanolic extracts of leaves from the indicated plant species were analyzed by FT-ICR MS for the occurrence of mass peaks m/z 293.124 and m/z 279.108 corresponding to glucoside conjugates of ILA and valic acid respectively.

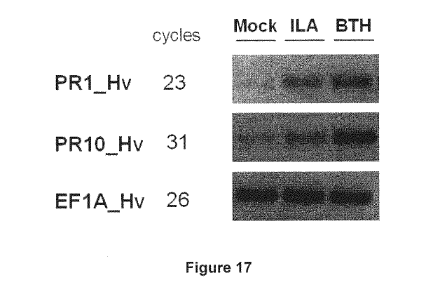

FIG. 17: Induction of defence genes in Hordeum vulgare (barley) leaves. Plants were sprayed with isoleucic acid, BTH or mock as described in Example 1 (Methods). Transcript levels of pathogen responsive genes PR1 and PR10 and the reference gene EF1A from barley (Hordeum vulgare, Hv) were monitored 48 h after treatment by semiquantitative PCR. The number of PCR amplification cycles is indicated.

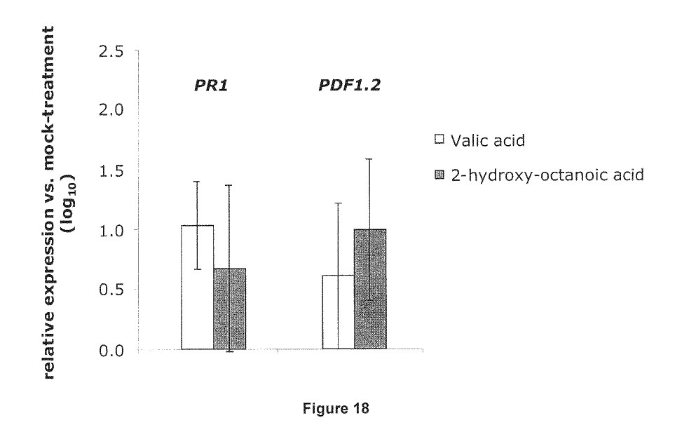

FIG. 18: Effects of exogenously applied octanoic acid and valic acid on the expression of defence marker genes PR1 and PDF1.2. Defence marker gene expression in 4-week-old Col-0 plants 24 h after treatment with 2-hydroxy-octanoic acid and valic acid (1 mM, dissolved in water). Expression levels were normalized to S16 transcript and expressed relative to the levels quantified for mock (water) treated plants. Chemicals were obtained from Sigma Aldrich. The graph shows preliminary results obtained from two biological replicates (means and standard deviations) indicating induction of PR1 and PDF1.2.

The examples illustrate the invention:

### EXAMPLE 1

### Methods and Materials

### Plant Materials and Growth Conditions

For infection experiments, RTqPCR analysis and plant transformation, plants were grown on soil (Floraton 1, Floragard, Germany) under an 12-14 h light cycle at 45 μmol m² s⁻¹ of light intensity at 18° C. in the dark and 20° C. in the light. For metabolic analysis of root material, plants were grown hydroponically at 120 μmol m² s⁻¹ of light intensity. Seeds were surface sterilized and grown on plates with ½ Murashige & Skoog medium including vitamins, 1% sucrose and 0.25% Gelrite. Seedlings were transplanted after 7 days in a floating hydroponic system (Battke et al., 2003) and grown for two more weeks. Each Vitro Vent box contained, 300 mL liquid medium and 250 mL polypropylene granulate as the floating material.

### Chemicals

Compounds A, E and F and valic acid ((S)-(+)-2-hydroxy-3-methylbutyric acid) were obtained from Sigma-Aldrich (Germany) and compound B from Interchim (France). Compounds C and D were not commercially available. Therefore, they were synthesized according to previously described protocols (Yabuuchi and Kusumi, 1999; Caille et al., 2009).

### Real-Time Quantitative RT-PCR

Rosette leaves of the indicated age were collected. Total RNA was isolated using the RNeasy Plant Mini Kit (Qiagen, Germany). RNA integrity and amount was analyzed by gel electrophoresis and by spectrophotometry. One microgram of total RNA was reverse transcribed using a SuperScript II reverse transcription-PCR kit (Invitrogen; Germany) according to the manufacturer's instructions. Gene-specific primer pairs were designed using the Primer Express 3.0 software. Primer pairs are listed in Table 1 below. All primer pairs were checked for amplification specificity and an efficiency superior to 80% using a serial cDNA dilution. Real time quantification was performed using a 7500 real time PCR system (Applied Biosystems, Germany). Individual PCR reaction mixtures contained 4 μL of diluted cDNA, 10 μL of Sybr Green Mastermix (Thermo Scientific, Germany) and 250 μM of each primer in a final volume of 20 μL. In all experiments, three biological replicates of each sample and two technical (PCR) replicates were performed. The amount of target gene was normalized over the abundance of the constitutive UBQ5 (At3g62250) and S16 (At5g18380, At2g09990) genes. The stability of the reference genes was tested and normalization was performed using GeNorm (Vandesompele et al., 2002). For RTqPCR of infected material, plants were infected as described below. Three biological replicates were analyzed; each consisted of six individually infected leaves. Plant material was harvested before infection and mock treatments (time point 0) and at the indicated time points after treatment. Each experiment was repeated with similar results.

For marker gene analysis on uninfected material and senescent leaves, methods for paired or grouped data were applied, namely the paired t-test and repeated measurements ANOVA (linear mixed-effects models), in order to control for interpolate variation (each replicate was measured on a different qPCR plate). Twoway ANOVA was used to join results from two independent analyses (three replicates each). First a model with interaction was fitted. If the interaction effect was significant, one-way ANOVAs were performed for the single experiments; otherwise a two-way ANOVA without interaction effect was fitted. All analyses (p-value, arithmetic mean) were performed on log₁₀-transformed data as recommended in the literature (Rieu and Powers, 2009). For all calculations, the R software with the nlme package was used (Pinheiro et al., 2009).

### Semiquantitative PCR Analysis

Total RNA was isolated using the RNeasy Plant Mini Kit (Qiagen, Germany). RNA integrity and amount was analyzed by gel electrophoresis and by spectrophotometry. One microgram of total RNA was reverse transcribed using a QuantiTect reverse transcription-PCR kit (Qiagen; Germany) according to the manufacturer's instructions. The following gene-specific primer pairs were kindly provided by Corina Vlot: EF1A_Hv_f (5′-GTCATTGATGCTCCTGGTCA-3′) and EF1A_Hv_r (5′-CTGCTTCACACCAAGAGTGA-3′) for the barley reference gene EF1A; PR1_Hv_f (5′-CGAGAAGAAGGACTACGACT-3′) and PR1_Hv_r (5′-CTCGTAGTTGCAGGTGATGA-3′) for barley PR1; Hv, PR10_Hv_f (5′-GTGGTCGAGATGAAGCTTGA-3′) and PR10_Hv_r (5′-ATCTTGAGCTGGTCGAGGTA-3′) for barley PR10. PCR fragments were amplified using the indicated number of PCR cycles (FIG. 17) and separated on a 2% agarose gel containing ethidium bromide.

UGT76B1 Overexpression and ugt76b1 Knockout Lines

Two T-DNA insertion lines in two different wt backgrounds, SAIL_{—}1171A11 [Col-0] and GT_{—}5_{—}11976 [Ler], were obtained from the NASC stock center (University of Nottingham, UK). The position of the T-DNA insert was confirmed by PCR and DNA sequencing using primers and SAIL_L (5′-TTCATAACCAATCTCGATACAC-3′) and 76B1_ORF_r (5′-GTCTGATTATGGGAATGCAGATTA-3′) for SAIL and primers 76B1_f620 (5′-AAGATCCAAGATCAGGGGATAAG-3′) and Ds5-2 mod (5′-CGTTTTGTATATCCCGTTTCCGT-3′) for GT-5. Lines were backcrossed once with their respective parental wild-type line and self-pollinated. Homozygous plants were identified by PCR, by amplification of the mutant allele using the same primers used for PCR and sequencing and by the absence of amplification of the wild-type allele using UGT76B1 specific primers 76B1_f620 and 76B1_ORF_r. Lack of the functional transcript in both knockout lines was confirmed by RT-PCR using the same gene specific primers. Two lines, named ugt76b1-1 and ugt76b1-2 isolated from SAIL_{—}1171A11 and GT_{—}5_{—}11976, respectively, were used for further experiments. Segregation of the F2 generation of both knockout lines after backcrossing was analyzed using the respective resistance markers.

UGT76B1 overexpression lines were produced by Agrobacterium-mediated transformation according to the state-of-the-art well known to experts using two different plasmid constructs pB2GW7 and pAlligator2 carrying the ORF coupled to a CaMV 35S-derived promoters (Bensmihen et al., 2004; Karimi et al., 2002). The following primers were used for UGT76B1 amplification and cloning using GATEWAY™ (Invitrogen, Germany) recombination: 5′-GGGGACAAGTTTGTACAAAAAAGCAGGCTA CACAATGGAGACTAGAGAAACAAAACCA-3′ and 5′-GGGGACCACTTTGTACAAGAAAGCTGGGTCT GATTATGGGAATGCAGATTA-3′. After selection of transformants segregation analysis was used for identification of single insertion lines in the T2 generation.

### Bacterial Strains and Determination of Bacterial Growth in Plants

Bacterial strains used in this study include Pseudomonas syringae pv. tomato DC3000 (Ps), P. syringae pv. tomato DC3000 (avrRpm1) (Ps-avir). Bacteria were grown overnight at 28° C. in King's B medium with appropriate antibiotics and diluted to 5*10⁵ cfu mL⁻¹ with 10 mM MgCl₂ for plant inoculation. Whole leaves of 5- to 6-week-old plants were infiltrated using a 1-mL syringe without a needle. Control plants were infiltrated with 10 mM MgCl₂. Leaf discs from control treated and infected plants were harvested from inoculated leaves at 0, 1 and 3 d after infiltration. Bacterial growth was assessed as described previously (Katagiri et al., 2002). For each time point, three samples were made by pooling six leaf discs from three different treated plants.

Salicylic acid Determination

Metabolites of pooled five-week-old rosette leaves from four to six individual plants (snap-frozen in liquid nitrogen and stored at −80° C.) were extracted with a 1+2 mixture of methanol and 2% (v/v) formic acid. The extract was split into three aliquots for separate determination of free SA, SA glucosides, and SA esters. For determination of the SA conjugates, the extract was digested overnight with β-glucosidase (Roth, Germany, cat. no. 7512.2) or with esterase (Sigma, Germany, cat. no. E2884). SA from undigested and digested samples was extracted under acidic conditions using reversed-phase sorbent cartridges (Oasis HLB 1 cc, Waters, WAT094225), recovered under basic conditions, and subsequently analyzed via HPLC. Quantification was based on SA fluorescence (excitation 305 nm/emission 400 nm) with o-anisic acid added as an internal standard during metabolite extraction and authentic SA standards. Thus, the content in free SA, in glucose-conjugated SA, and in esterified SA could be acquired.

### Non-Targeted Metabolome Analysis

Three biological replicates and two technical replicates each were used for each genotype. Frozen root tissue was individually disrupted using a dismembrator. Metabolite extraction was performed as described previously (Weckwerth et al., 2004) with slight modifications. 44 μg mL⁻¹ loganin and 3 μg mL⁻¹ nitrophenol were added to the extraction buffer (methanol/chloroform/water 2.5:1:1 v/v/v) as internal standards. After extraction the aqueous phase was divided in several 200 μL aliquots and dried completely using a Speedvac. For FT-ICR MS analysis one dried aliquot from each sample was redissolved in 70% methanol and diluted 1:25 in 70% methanol containing 35 pmol mL⁻¹ di-alanin.

Ultrahigh-resolution mass spectra were acquired on a Bruker APEX Qe Fourier Transform ion cyclotron resonance mass spectrometer FT-ICR MS (Brukers, Germany) equipped with a 12 Tesla superconducting magnet and an APOLLO II Electrospray ionization source. Measurements were performed in the negative ionization mode (see Supplemental Methods online).

Mass lists were calibrated using the Data Analysis program (Bruker, Germany) and exported to ascii files. Mass list matrices for statistical analysis were produced using a custom-made program (M. Frommberger, Helmholtz Center, Germany). Masses, which were detected in only two or less out of six measurements in both genotypes, were deleted. Pearson correlation analysis (excluding missing values) was used to check extract reproducibility (correlation r²>0.9). Sum of total peak intensities was monitored to detect variation in the ionization efficiency (additionally to internal standards). Non-detectable peaks were replaced by 200,000 counts, which were considered as the detection limit, to enable calculation of mean values and ratios. A two sample Wilcoxon rank sum test was performed for each mass separately to detect significant peak intensity differences between wild-type and mutant plants. Significance level was set to 1%. Measurements were repeated twice to filter for reproducible metabolite variations and only those peaks were selected, which correlated with UGT76B1 expression in both knockout and overexpression lines. Statistical analysis was performed in R.

For MS/MS fragmentation studies the plant extract from UGT76B1-OE-7 was partially cleaned and concentrated.

### Recombinant UGT76B1 and Glucosyltransferase Assay

A glutathione-S-transferase (GST)-UGT761 expression plasmid was constructed using pDEST15 (Invitrogen, Germany). The UGT76B1 open reading frame was amplified with the same primers as used for the construction of the overexpression lines. The recombinant protein was affinity-purified using a glutathione-coupled sepharose beads according to the manufacturer's instructions (GE Healthcare, Germany), concentrated by membrane filtration (Amicon Ultra-4; Millipore, Germany) and supplemented with 20% glycerol for storage at −20° C. (Messner et al., 2003).

To analyze the UGT enzyme activity assay mixtures contained 0.1 M Tris-HCl (pH 7.5), 5 mM UDP-glucose, 0.5 mM aglycone and about 1 μg fusion protein in a final volume of 50 μL. After incubation for 1 hour at 30° C. the reaction was stopped by addition of 200 μL methanol and cleared by centrifugation. Reactions were diluted 1:50 in 70% methanol (except for valic acid, which was used without dilution) and analyzed on a API4000 mass spectrometer using direct injection into the electrospray source at a flow rate of 30 μL. 150 Scans were accumulated for each measurement in dual ion monitoring mode, which was adjusted to monitor ions at nominal m/z ratios of the corresponding expected substrate and product peaks with a mass range of ±5 Da.

### Isoleucic Acid Treatment of Arabidopsis Plants

For isoleucic acid treatment, 4-week-old plants were sprayed with 0.5 or 1 mM isoleucic acid (diluted in water) or only water for mock treatments. Plants were covered with a plexiglass lid until the surface of the leaves became dry. The fifth to eighth true leaves of each plant were harvested 24 hours after treatment. Four leaves from three independent plants were pooled for each replicate and analyzed by real-time PCR.

### Isoleucic Acid and BTH Treatment of Barley Plants

For ILA treatment in barley, 2-week-old plants were sprayed with either 1 mM ILA or 0.25 mg/ml BTH (benzothiadiazole/BION™), dissolved in water with 0.01% tween. For mock treatment, plants were sprayed only with water containing 0.01% tween. Leaves were harvested 48 hours after treatment and analyzed by semiquantitative PCR.

### Data Acquisition for FT-ICR MS Analyses

Ultrahigh-resolution mass spectra were acquired on a Bruker APEX Qe Fourier Transform ion cyclotron resonance mass spectrometer FT-ICR MS (Brukers, Germany) equipped with a 12 Tesla superconducting magnet and an APOLLO II Electrospray ionization source. Measurements were performed in the negative ionization mode. Samples were introduced into the electrospray source at a flow rate of 120 μL/h with a nebulizer gas pressure of 20 psi and a drying gas pressure of 15 psi (at 200° C.). Spectra were externally calibrated based on arginine cluster ions (10 ppm). The spectra were acquired with a time domain of 1 MW over a mass range between 146 and 2000 amu. Three hundred scans were accumulated for each spectrum. Internal mass calibration was performed using the internal standards (loganin, dialanin) in addition to endogenous plant metabolites with calibration accuracy smaller than 0.01 ppm. Internal standards were also used to detect variation in the extraction procedure, matrix effects and variation in the ionisation efficiency in the Electrospray source.

### Fragmentation Studies Using FT-ICR MS

For MS/MS fragmentation studies, the plant extract from UGT76B1-OE-7 was partially cleaned and concentrated using a Strata NH2 column (3 mL, Phenomenex; Germany). The targeted ions were trapped in a first hexapole for 200 ms prior to their mass selection inside a quadrupole mass filter. Once isolated, the targeted ions were then accelerated and were let to collide with argon atoms inside a second hexapole which serves as a collision cell. The second hexapole had a relatively high pressure of 5×10-3 mbar. As a result of the collisions between the accelerated isolated ions and argon atoms in the second hexapole, product ions were produced and they were forwarded to the ICR cell via a couple of accelerating and decelerating lenses. The ion accumulation time inside the collision cell was 500 ms. For targeted ions with m/z<200 amu, no quadrupole MS/MS fragmentation was done. Instead, the ions were forwarded as normal to the ICR cell and then they were isolated inside the cell by applying a frequency sweep to eject all ions but those that should be selected for further fragmentation event. Once isolated inside the ICR cell, the targeted ions could be excited in the radial plane, which was perpendicular to the magnetic field lines by applying an on-resonance radial single shot excitation pulse with a duration of 400 μs and a power of 4.5 Vp-p. A pulsed valve opened at the same time for 5 ms to inject argon atoms inside the ICR cell for collisional induced dissociation experiments. The produced fragment ions were then allowed to thermalize inside the cell before accelerating them in the radial plane for detection.

TABLE 1
Primer pairs used for RTqPCR Analysis.

| | Accession | | | |
|---|---|---|---|---|
| Gene | number | Primer | Primer sequence | SEQ ID NO: |
| UBQ5 | At3g62250 | UBQ5_f | ggtgctaagaagaggaagaat | 1 |
| | | UBQ5_r | ctccttcttctggtaaacgt | 2 |
| S16 | At5g18380, | S16qRT_f | tttacgccatccgtcagagtat | 3 |
| | At2g09990 | S16qRT_r | tctggtaacgagaacgagcac | 4 |
| UGT76B1 | At3g11340 | 76B1 f | tggaagatcggattgcatt | 5 |
| | | 76B1_r | ccttcatgggcataatcctc | |
| PR1 | At2g14610 | PR1_f | gtgccaaagtgaggtgtaaca | |
| | | | a | 7 |
| | | PR1_r | cgtgtgtatgcatgatcacatc | 8 |
| PDF1.2 | At5g44420 | PDF1.2a_f (1) | ccaagtgggacatggtcag | 9 |
| | | PDF1.2a_r (1) | acttgtgtgctgggaagaca | 10 |
| VSP2 | At5g24770 | VSP2_f | ttggcaatatcggagatcaat | 11 |
| | | VSP2_r | gggacaatgcgatgaagatag | 12 |
| SAG13 | At2g29350 | SAG13_f | ttgcccacccattgttaaa | 13 |
| | | SAG13_r | gattcatggctcctttggtt | 14 |
| SAG12 | At5g45890 | SAG12 f | aatgatgagcaagcactgatg | 15 |
| | | SAG12_r | cgtagtgcactctccagtgaa | 16 |
| LOX2 | At3g45140 | LOX2_f (2) | tgcacgccaaagtcttgtca | 17 |
| | | LOX2_r (2) | tcagccaacccccttttga | 18 |
| WRKY70 | At3g56400 | WRKY70_f | ggaagaagacaatcctcatcgt | 19 |
| | | WRKY70_r | cgttttcccattgacgtaact | 20 |
| EDS1 | At3g48090 | EDS1_f (3) | cgaagacacagggccgta | 21 |
| | | EDS1_r (3) | aagcatgatccgcactcg | 22 |
| PAD4 | At3g52430 | PAD4_f (3) | ggttctgttcgtctgatgttt | 23 |
| | | PAD4_r (3) | gttcctcggtgttttgagtt | 24 |

(1) Primers were previously described in Kumar et al. 2009; (2) Primers were previously described in Delker et al. 2007; (3) Primers were previously described in Straus et al. 2010.

### EXAMPLE 2

### ugt76b1 Knockout and UGT76B1 Over-Expression Lines

To study whether UGT76B1 has any function in plant stress responses and how this might affect the plant, we obtained Arabidopsis loss-of-function mutants and generated constitutive over-expression lines of UGT76B1. Two T-DNA insertion lines SAIL_{—}1171A11 and GT_{—}5_{—}11976 in two different genetic backgrounds (Col-0 and Ler) were characterized as ugt76b1-1 and ugt76b1-2 knockout mutants, respectively. Sequencing confirmed the position of the insertions. Also, a 3:1 segregation after backcrossing verified that the mutation was inherited as a single locus in both cases.

RT-PCR analysis confirmed the lack of UGT76B1 transcripts in both lines (see FIG. 9). Arabidopsis lines overexpressing UGT76B1 under the control of 35S-derived constitutive promoters were generated and two homozygous lines with single insertions were selected (see Methods). Both lines, UGT76B1-OE-5 and UGT76B1-OE-7 showed a significantly higher transcript level compared to the wild type (see FIG. 9).

### EXAMPLE 3

### UGT76B1 Over-Expression and Loss-of-Function Alter Pathogen Susceptibility in an Opposite Manner

As changing UGT76B1 expression had no influence on plant resistance to abiotic stressors like UV irradiation and salt, we checked whether alterations in UGT76B1 expression would affect the susceptibility of the plant to biotrophic pathogens. Whole leaves of ugt76b1-1, UGT76B1-OE-7 and Col-0 were inoculated with 5·10⁵ cfu mL⁻¹ avirulent Pseudomonas syringae strain D3000 AvrRpt2 (Ps-avir). The bacteria showed the typical proliferation of Ps-avir in Col-0 30 h and 78 h after inoculation. In the knockout plant, nearly no bacterial growth was observed pointing to a significantly reduced susceptibility, whereas in the over-expression line the bacterial population strongly increased indicating a reduced resistance (FIG. 1). Similar results were obtained with virulent Pseudomonas syringae DC3000 (Ps-vir). The ugt76b1-1 knockout mutant showed a strongly reduced bacterial growth, whereas plant susceptibility was increased in the over-expression line (FIG. 1). In both cases, UGT76B1 expression negatively correlated with plant resistance.

### EXAMPLE 4

### Defense Marker Gene Expression is Constitutively Altered in UGT76B1-OE and ugt76b1 Lines

As gain-of-resistance mutants may show constitutively enhanced transcript levels of defense-response genes, we analyzed several defense marker genes in UGT76B1-OE-7 and ugt76b1-1 lines using relative quantification by RTqPCR. PAD4 and EDS1 act upstream from SA biosynthesis, but are also induced by SA. PR1 is a pathogen and SA responsive gene, which is a well established marker gene for the defense responses of Arabidopsis against Pseudomonas (reviewed in Vlot et al., 2009). SAG13 is an early senescence marker, which is also induced by several stress factors and SA (Weaver et al., 1998). WRKY70 encodes a transcription factor and is an important regulator in the interplay of SA- and JA-related plant defense responses (reviewed in Vlot et al., 2009). PDF1.2 and VSP2 are marker genes frequently used to monitor JA and ethylene responses (Pieterse et al., 2009), whereas LOX2 involved in JA biosynthesis is activated by a positive feedback loop (Sasaki et al., 2001). Changing UGT76B1 expression had a strong effect on the transcript level of these defense-related genes (FIG. 2A). PR1, PAD4, EDS1, WRKY70 and SAG13 were induced in leaves of five-week-old untreated ugt76b1 knockout plants compared to the wild type. In contrast, JA-responsive genes PDF1.2 and VSP2 as well as LOX2 were down-regulated. UGT76B1-OE-7 showed the opposite regulation for all measured genes. PR1, PAD4, EDS1, WRKY70 and SAG13 were downregulated, whereas VSP2 and LOX2 were up-regulated. The up-regulation of PDF1.2 in UGT76B1-OE-7 was more variable in different experiments. To exclude an age-dependent effect on defense gene expression (Kus et al., 2002), PR1 and SAG13 were analyzed also in younger, three-week-old plants. Both genes showed a similar, opposite regulation in knockout and over-expression lines (see FIG. 10).

To analyze whether the over-expression line was able to induce SA-dependent defense after Pseudomonas challenge, although it was compromised by the strong constitutive repression of this pathway, we analyzed the transcription of PR1 and SAG13 in wild-type, mutant and UGT76B1-overexpressing plants after bacterial inoculation. In wild-type plants PR1 and SAG13 were induced 24 h after infection with Ps-avir to similar levels as those constitutively expressed in the ugt76b1 loss-of-function mutant. In the overexpression line, expression of both PR1 and SAG13 reached similar levels as in wild-type plants 24 h after pathogen challenge. Thus, the general ability to perceive and respond to the pathogen was not altered in UGT76B1-OE-7 (FIG. 2B).

### EXAMPLE 5

### Endogenous Levels of Free and Conjugated SA are Elevated in ugt76b1

EDS1 and PAD4 are essential regulators of basal resistance and are known to control the accumulation of the signaling molecule salicylic acid. In addition, several gain-of-resistance mutants with transcriptional activation of PR genes are known to have increased levels of SA and its glucosides (reviewed in Vlot et al., 2009) We therefore assessed whether the high level of defense gene expression in ugt76b1-1 plants was correlated with higher endogenous SA levels (FIG. 3). Indeed ugt76b1-1 showed a considerably higher basal level of SA and its glucosides than detected in wild-type plants in the absence of any inducer. In contrast, the overexpression line contained an amount of free SA that was similar to that in wild-type plants showing even a tendency for repression, but curiously also higher levels of the SA conjugate. The SA ester level did not significantly change in overexpression lines, but was slightly increased in the knockout mutant (FIG. 3).

### EXAMPLE 6

### UGT76B1 is Induced Early after Pathogen Infection Prior to PR1

To know at which time point after pathogen infection UGT76B1 transcription was activated, we analyzed the time course of UGT76B1 expression after pathogen infection compared to other defense marker genes known to be induced at early or late phases during the defense response. FIG. 4 shows the time course of UGT76B1, SAG13, WRKY70, EDS1, PAD4 and PR1 expression during the incompatible interaction of wild-type plants with Ps-avir. PR1 as well as SAG13 were highly induced 24 h after pathogen inoculation. UGT76B1 as well as WRKY70, EDS1 and PAD4 clearly preceded the upregulation of PR1 and SAG13.

### EXAMPLE 7

### Non-Targeted Metabolome Analysis Reveals Correlation Between Isoleucic Acid Glucoside Formation and UGT76B1 Expression

In the case of the broadly stress-inducible UGT76B1 gene, co-expression analyses did not indicate an assignment, which could hint towards a class of potential substrates. Thus, we aimed at using a non-targeted approach to obtain information on the affected pathway or substrate without any other prior knowledge. An ultrahigh-resolution 12 Tesla FT-ICR mass spectrometer run in the negative ionization mode was employed to compare the metabolic profile of UGT76B1-OE and ugt76b1 mutants with their respective wild type. Root material from plants grown in hydroponic culture was used as a starting material for metabolite extraction, because UGT76B1 was mainly expressed in roots and showed only lower expression in leaves under unstressed conditions. A stringent, combinatorial screening for metabolite changes was performed across the two independent knockout lines in two different wild-type backgrounds and both independent over-expression lines. By setting a p-value cut-off smaller than 0.01 and by filtering for metabolites, which showed consistent and opposite regulation in knockout and over-expression plants (in different accessions as background), only two metabolites were found whose accumulation was significantly and positively correlated with UGT76B1 expression. Both m/z peaks were repressed in the knockout and induced in the over-expression lines (FIG. 5A; Methods). In addition, both peaks were significantly enhanced as compared to wild-type in leaf material of the UGT76B1 over-expression lines, although with an overall lower intensity than in roots (see FIG. 11). Thus, this combinatorial approach allowed to pinpoint informative molecules from the non-targeted metabolome analyses.

Due to the high accuracy in m/z determination, an exact molecular formula could be assigned to both peaks. Fragmentation studies further revealed that the molecule with m/z 293 was a glucoside (FIG. 5B), This highly suggested that it indicated the in planta product of UGT76B1. No glucoside loss could be observed upon fragmentation of the second peak (m/z 279). Loss of the glucosidic moiety from m/z 293 led to a smaller compound with m/z 131. The molecular formula of this residual aglycon was C₆H₁₂O₃. Further in-cell fragmentation of this molecule led to the loss of a formic acid (CH₂O₂) moiety and the formation of a second fragment m/z 85. According to a previous study this behavior indicated that the aglycon of m/z 293 was an α-hydroxy carboxylic acid with a free β-hydrogen (Bandu et al., 2006). Thus, six possible structures could be suggested for the aglycon m/z 131 (FIG. 5C). Structures A, C, D, and F could be excluded, because the fragmentation of the corresponding standard compounds gave rise to further fragments, which were not detected after fragmentation of the unknown aglycon from the plant extract (m/z 131) (see FIG. 12). Both compounds B and E gave the same fragmentation pattern as the unknown plant peak and therefore constituted possible candidate structures of the aglycon.

### EXAMPLE 8

### In Vitro Activity of Recombinant UGT76B1 Towards Isoleucic Acid

In order to further elucidate the structure of m/z 131, compounds B and E were tested as potential substrates of recombinant UGT76B1 in vitro. As shown in FIG. 6C, UGT76B1 glucosylated isoleucic acid (compound B, 2-hydroxy-3-methylpentanoic acid), whereas it showed no activity towards 2-ethyl-2-hydroxybutyric acid (compound E, see FIG. 13). Thus, isoleucic acid turned out to be a substrate of UGT76B1 in vitro, which was in accordance with the observation of plant extracts derived from ugt76b1 knockout and UGT76B1-OE lines.

The UGT76B1-dependent formation of isoleucic acid (ILA) glucoside negatively correlated with pathogen resistance and onset of senescence. Neither isoleucic acid nor its glucoside have been described before in Arabidopsis. The second compound with m/z 279 (C₁₁H₂₀O₈) found to be correlated with UGT76B1 expression in our non-targeted metabolomics approach differed from the isoleucic acid-glucoside peak (m/z 293, C₁₂H₂₂O₈) by one CH₂ moiety. Therefore, it could represent the corresponding glucosylated compound derived from valine metabolism (2-hydroxy-3-methylbutyric acid, valic acid). Although MS/MS fragmentation did not yield cleavage of a glucose moiety, we nevertheless checked the in vitro activity of the recombinant enzyme towards this compound (FIG. 6 D-F). The results indicate that UGT76B1 is also able to glucosylate valic acid.

Amino acid-derived molecules have also been related to Arabidopsis defense reactions by the involvement of two aminotransferases ALD1 and AGD2, which supposedly catalyze an amino transfer in opposite directions acting on an unknown α-keto acid/α-amino acid couple (Song et al., 2004). The authors found that agd2 mutants were more resistant to Pseudomonas syringae infection, while ald1 plant showed increased susceptibility.

### EXAMPLE 9

### Direct Effect of Isoleucic Acid on Defense Mechanisms

The identification of isoleucic acid as a substrate of UGT76B1 raised the question whether isoleucic acid itself was an active compound in planta. Indeed, exogenously applied isoleucic acid strongly affects plant defense pathways. Twenty-four hours after spraying an isoleucic acid solution onto leaves of four-week-old plants, PR1 expression was more than tenfold induced showing a direct and positive influence on the SA pathway. In contrast, the JA-marker genes VSP2 and PDF1.2 were not significantly influenced; while VSP2 was only marginally suppressed, PDF1.2 showed a tendency for induction, but was highly variable as well (FIG. 7).

Since ILA induced the defence marker gene PR1, it was analysed whether this translated into an enhanced resistance toward P. syringae. Indeed, plants that had been treated with ILA before infection by Ps-avir showed about four- to fivefold less bacterial growth compared with mock treatment (FIG. 14). The increased resistance was persistent for at least 1 to 3 d after ILA spraying. Optimization of the spraying regime and/or the use of surfactants might further enhance the protective effect.

### EXAMPLE 10

### Effect of Other α-Hydroxy-Acids on Plant Defence Mechanisms

As shown in FIG. 15, other tested α-hydroxy-acids show similar or even stronger induction of defence marker genes PR1 and PDF1.2 as compared to ILA.

### EXAMPLE 11

### Valic Acid- and ILA-Glucosides in Other Plant Species

To analyze weather α-hydroxy-acids might also have similar functions in other plant species, several plants were analyzed for the occurrence of the mass peaks corresponding to valic acid- and ILA glucosides. As shown in FIG. 16, both peaks could be detected in a wide range of dicotyledonous as well as monocotyledonous plants. Although these peaks were not further identified, they suggest the occurrence of valic acid- and ILA glucosides in the analyzed plants.

### EXAMPLE 12

### ILA Induces Pathogen Responsive Genes in the Monocotyledonous Plant Hordeum vulgare (Barley)

Since mass peaks corresponding to valic acid and ILA-glucosides could also be detected in barley, it was analysed whether ILA could also induce defense related genes in barley. Treatment with BTH (benzothiadiazole), a salicylic acid (SA) analog, was used as a positive control. Expression levels of the barley reference gene EF1A served as an internal control. As shown in FIG. 17, ILA induces transcript levels of two different pathogen responsive genes in barley, PR1 and PR10. This indicates that treatment with α-hydroxy-acids can also increase pathogen resistance in monocotyledonous plant species.

### EXAMPLE 13

### Effects of α-Hydroxy-Acids with Different Chain Length on Plant Defence Mechanisms

As shown in FIG. 18, preliminary results indicate that 2-hydroxy-octanoic acid and valic acid can also induce the expression of defense marker genes when exogenously applied to the plant.

### REFERENCES

Bandu, M. L., Grubbs, T., Kater, M., and Desaire, H. (2006). Collision induced dissociation of alpha hydroxy acids: Evidence of an ion-neutral complex intermediate. International Journal of Mass Spectrometry 251: 40-46.
Battke, F., Schramel, P., and Ernst, D. (2003). A novel method for in vitro culture of plants: Cultivation of barley in a floating hydroponic system. Plant Molecular Biology Reporter 21: 405-409.
Bensmihen, S., To, A., Lambert, G., Kroj, T., Giraudat, J., and Parcy, F. (2004). Analysis of an activated ABI5 allele using a new selection method for transgenic Arabidopsis seeds. FEBS Lett 561: 127-131.
Bolton, M. D. (2009). Primary metabolism and plant defense—fuel for the fire. Mol Plant Microbe Interact 22: 487-497.
Bowles, D., Lim, E. K., Poppenberger, B., and Vaistij, F. E. (2006). Glycosyltransferases of lipophilic small molecules. Annual Review of Plant Biology 57: 567-597.
Caille, S., Cui, S., Hwang, T. L., Wang, X., and Faul, M. M. (2009). Two asymmetric syntheses of AMG 221, an inhibitor of 11 beta-hydroxysteroid dehydrogenase type 1. J Org Chem 74: 3833-3842.
Dean, J. V., and Delaney, S. P. (2008). Metabolism of salicylic acid in wild-type, ugt74f1 and ugt74f2 glucosyltransferase mutants of Arabidopsis thaliana. Physiol Plant 132: 417-425.
Delker, C., Zolman, B. K., Miersch, O., and Wasternack, C. (2007). Jasmonate biosynthesis in Arabidopsis thaliana requires peroxisomal beta-oxidation enzymes—Additional proof by properties of pex6 and aim1. Phytochemistry 68: 1642-1650.
Fonseca, S., Chini, A., Hamberg, M., Adie, B., Porzel, A., Kramell, R., Miersch, O., Wasternack, C., and Solano, R. (2009). (+)-7-iso-Jasmonoyl-L-isoleucine is the endogenous bioactive jasmonate. Nat Chem Biol 5: 344-350.
Fridman, E., and Pichersky, E. (2005). Metabolomics, genomics, proteomics, and the identification of enzymes and their substrates and products. Curr Opin Plant Biol 8: 242-248.
Garcia, A., Servane Blanvillain-Baufumé, S., Robin P. Huibers, R P., Marcel Wiermer, M., Li, G., Gobbato, E., Steffen Rietz, S., Parker, J E. (2010). Balanced nuclear and cytoplasmic activities of EDS1 are required for a complete plant innate immune response. PLoSPath (in press).
Hanson, A. D., Pribat, A., Waller, J. C., and de Crecy-Lagard, V. (2010). ‘Unknown’ proteins and ‘orphan’ enzymes: the missing half of the engineering parts list—and how to find it. Biochemical J 425: 1-11.
Heil, M., and Baldwin, I. T. (2002). Fitness costs of induced resistance: emerging experimental support for a slippery concept. Trends Plant Sci 7: 61-67.
Hirai, M. Y., Klein, M., Fujikawa, Y., Yano, M., Goodenowe, D. B., Yamazaki, Y., Kanaya, S., Nakamura, Y., Kitayama, M., Suzuki, H., Sakurai, N., Shibata, D., Tokuhisa, J., Reichelt, M., Gershenzon, J., Papenbrock, J., and Saito, K. (2005). Elucidation of gene-to-gene and metabolite-to-gene networks in arabidopsis by integration of metabolomics and transcriptomics. J Biol Chem 280: 25590-25595.
Jones, J. D., and Dangl, J. L. (2006). The plant immune system. Nature 444: 323-329.
Jones, P., and Vogt, T. (2001). Glycosyltransferases in secondary plant metabolism: tranquilizers and stimulant controllers. Planta 213: 164-174.
Karimi, M., Inze, D., and Depicker, A. (2002). GATEWAY™ vectors for Agrobacterium-mediated plant transformation. Trends Plant Sci 7: 193-195.
Katagiri, F., Thilmony, R., and He, S. (2002). The Arabidopsis thaliana-Pseudomonas syringae interaction. In The Arabidopsis Book, C. R. Somerville, Meyerowitz, E. M., eds., Rockville, M.D.: Am. Soc. Plant Biologists, pp. 1-35.
Koornneef, A., and Pieterse, C. M. (2008). Cross talk in defense signaling. Plant Physiol 146: 839-844.
Kumar, M., Busch, W., Birke, H., Kemmerling, B., Nümberger, T., and Schöffl, F. (2009). Heat Shock Factors HsfB1 and HsfB2b Are Involved in the Regulation of Pdf1.2 Expression and Pathogen Resistance in Arabidopsis. Molecular Plant 2: 152-165.
Mackenzie, P. I., Owens, I. S., Burchell, B., Bock, K. W., Bairoch, A., Belanger, A., Fournel-Gigleux, S., Green, M., Hum, D. W., Iyanagi, T., Lancet, D., Louisot, P., Magdalou, J., Chowdhury, J. R., Ritter, J. K., Schachter, H., Tephly, T. R., Tipton, K. F., and Nebert, D. W. (1997). The UDP glycosyltransferase gene superfamily: recommended nomenclature update based on evolutionary divergence. Pharmacogenetics 7: 255-269.
Mamer 2000, Methods in Enzymology, volume 324, pages 3 to 10
Matsuda, F., Yonekura-Sakakibara, K., Niida, R., Kuromori, T., Shinozaki, K., and Saito, K. (2009). MS/MS spectral tag-based annotation of non-targeted profile of plant secondary metabolites. Plant J 57: 555-577.
Messner, B., Thulke, O., and Schäffner, A. R. (2003). Arabidopsis glucosyltransferases with activities toward both endogenous and xenobiotic substrates. Planta 217: 138-146.
Ohta, D., Kanaya, S., and Suzuki, H. (2010). Application of Fourier-transform ion cyclotron resonance mass spectrometry to metabolic profiling and metabolite identification. Curr Opin Biotechnol 21: 35-44.
Pieterse, C. M., Leon-Reyes, A., Van der Ent, S., and Van Wees, S. C. (2009). Networking by small-molecule hormones in plant immunity. Nat Chem Biol 5: 308-316.
Pinheiro, J., Bates, D., DebRoy, S., Sarkar, D., and team., t. R. C. (2009). nlme: Linear and Nonlinear Mixed Effects Models. R package version 3.1-94.
Rieu, I., and Powers, S. J. (2009). Real-Time Quantitative RT-PCR: Design, Calculations, and Statistics. Plant Cell 21: 1031-1033.
Saito, K., Hirai, M. Y., and Yonekura-Sakakibara, K. (2008). Decoding genes with coexpression networks and metabolomics—‘majority report by precogs’. Trends Plant Sci 13: 36-43.
Sasaki, Y., Asamizu, E., Shibata, D., Nakamura, Y., Kaneko, T., Awai, K., Amagai, M., Kuwata, C., Tsugane, T., Masuda, T., Shimada, H., Takamiya, K., Ohta, H., and Tabata, S. (2001). Monitoring of methyl jasmonate-responsive genes in Arabidopsis by cDNA macroarray: self-activation of jasmonic acid biosynthesis and crosstalk with other phytohormone signaling pathways. DNA Res 8: 153-161.
Song, J. T., Lu, H., and Greenberg, J. T. (2004). Divergent roles in Arabidopsis thaliana development and defense of two homologous genes, aberrant growth and death2 and AGD2-LIKE DEFENSE RESPONSE PROTEIN1, encoding novel aminotransferases. Plant Cell 16: 353-366.
Song, J. T., Koo, Y. J., Seo, H. S., Kim, M. C., Choi, Y. D., and Kim, J. H. (2008). Overexpression of AtSGT1, an Arabidopsis salicylic acid glucosyltransferase, leads to increased susceptibility to Pseudomonas syringae. Phytochemistry 69: 1128-1134.
Snowden, R. L., Grenno, R, and Vial, C. (2005). Synthesis and organoleptic properties of various 3-methyl-2-oxopentanoates, 2-hydroxy-3-methylpentanoates, and 2-acyloxy-3-methylpentanoates. Flavour Fragr. J. 20: 372-380.
Straus, M. R., Rietz, S., van Themaat, E. V., Bartsch, M., and Parker, J. E. (2010). Salicylic acid antagonism of EDS1-driven cell death is important for immune and oxidative stress responses in Arabidopsis. Plant J (in press).
Vandesompele, J., De Preter, K., Pattyn, F., Poppe, B., Van Roy, N., De Paepe, A., and Speleman, F. (2002). Accurate normalization of real-time quantitative RT-PCR data by geometric averaging of multiple internal control genes. Genome Biol 3: RESEARCH0034.1-0034.11.
Vlot, A. C., Dempsey, D. A., and Klessig, D. F. (2009). Salicylic Acid, a multifaceted hormone to combat disease. Annu Rev Phytopathol 47: 177-206.
Vogt, T., and Jones, P. (2000). Glycosyltransferases in plant natural product synthesis: characterization of a supergene family. Trends Plant Sci 5: 380-386.
Weaver, L. M., Gan, S. S., Quirino, B., and Amasino, R. M. (1998). A comparison of the expression patterns of several senescence-associated genes in response to stress and hormone treatment. Plant Molecular Biology 37: 455-469.
Weckwerth, W., Wenzel, K., and Fiehn, O. (2004). Process for the integrated extraction identification, and quantification of metabolites, proteins and RNA to reveal their co-regulation in biochemical networks. Proteomics 4: 78-83.
Yabuuchi, T., and Kusumi, T. (1999). A convenient one-pot synthesis of quaternary alpha-methoxy- and alpha-hydroxycarboxylic acids. Chemical & Pharmaceutical Bulletin 47: 684-686.
Yonekura-Sakakibara, K., Tohge, T., Matsuda, F., Nakabayashi, R., Takayama, H., Niida, R., Watanabe-Takahashi, A., Inoue, E., and Saito, K. (2008). Comprehensive flavonol profiling and transcriptome coexpression analysis leading to decoding gene-metabolite correlations in Arabidopsis. Plant Cell 20: 2160-2176.

1. A plant protective composition comprising α-hydroxy-acid or a derivative thereof.

2. The plant protective composition of claim 1, wherein the α-hydroxy-acid is represented by the general formula (I): 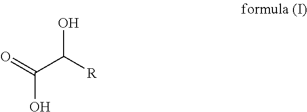
wherein R is hydrogen or a linear or branched C1 to C6 alkyl group.

3. The plant protective composition of claim 1, wherein the α-hydroxy-acid is 2-hydroxy-3-methyl-pentanoic acid.

4. The plant protective composition of claim 1, wherein the derivative is selected from the group consisting of an ester or an anhydride of an α-hydroxy-acid.

5. The plant protective composition of claim 1, wherein the composition further comprises a carrier and/or additive.

6. The plant protective composition of claim 1, wherein the composition is selected from the group consisting of directly sprayable solutions, dilutable solutions, aqueous solutions, emulsifiable concentrates, coatable pastes, dilute emulsions, wettable powders, soluble powders, dusts, granulates, encapsulations, natural substances impregnated with active compound and synthetic substances impregnated with active compound.

7. The plant protective composition of claim 1, wherein the concentration of the α-hydroxy-acid or derivative thereof in the composition is between 1 μM and 2 mM.

8. The plant protective composition of claim 1, wherein treatment with the composition reduces the stress-induced damage of plant tissue or the amount of pathogens present in the plant to less than 50% of the damage or amount of pathogens found in plants not treated with the protective composition.

9. The plant protective composition of claim 1, wherein the composition protects the plant against pathogens.

10. The plant protective composition of claim 9, wherein the pathogens are selected from the group consisting of bacteria, fungi and viruses.

11. The plant protective composition of claim 1, wherein the composition induces an endogenous plant resistance mechanism.

12. The plant protective composition of claim 1, wherein the plant is selected from the group consisting of monocotyledonous plants and dicotyledonous plants.

13. A method of protecting plants from pathogens comprising contacting the plants with the plant protective composition of claim 1.

14. The method of claim 13, wherein the plants are contacted by any one selected from the group consisting of spraying, dusting, scattering, coating and pouring.
